(19) United States
(12) Patent Application Publication
(10) Pub. No.: US 2021/0161855 A1
(43) Pub. Date: Jun. 3, 2021

(54) 5-LIPOXYGENASE ANTAGONISTS

(71) Applicant: INSTITUTO DE MEDICINA MOLECULAR JOÃO LOBO ANTUNES, 1649-028 Lisbon (PT)

(72) Inventors: Gonçalo BERNARDES, 1649-028 Lisbon (PT); Tiago RODRIGUES, 1649-028 Lisbon (PT)

(21) Appl. No.: 16/645,126

(22) PCT Filed: Sep. 6, 2018
(86) PCT No.: PCT/PT2018/050029

§ 371 Date: Mar. 6, 2020

(30) Foreign Application Priority Data
Sep. 8, 2017 (GB) 17144965

(51) Int. Cl. A61K 31/352; A61P 35/02; A61K 31/122; A61K 31/343; A61K 31/7056; A61K 31/704; A61K 31/7068; A61K 31/475; A61K 38/50; A61K 31/675; A61K 31/519; A61K 31/52; A61K 39/395; A61K 31/337; A61K 31/196; A61K 45/06

(52) U.S. Cl. CPC A61K 31/352; A61K 45/06; A61K 31/122; A61K 31/343; A61K 31/7056; A61K 31/704; A61K 31/7068; A61K 31/475; A61K 38/50; A61K 31/675; A61K 31/519; A61K 31/52; A61K 39/3955; A61K 31/337; A61K 31/196; A61P 35/02

(57) ABSTRACT

This invention relates to the treatment of conditions, such as cancer, associated with 5-lipoxygenase (5-LO) expression using β-lapachone compounds that inhibit 5-lipoxygenase (5-LO), such as β-lapachone and derivatives thereof. Methods of treatment of conditions associated with 5-lipoxygenase (5-LO) expression as well as medical uses of β-lapachone compounds in such methods are provided, as well as methods of selecting or prognosing cancer patients.

### FIELD

This invention relates to the inhibition of 5-lipoxygenase using β-lapachone compounds. This may be useful for example in the treatment of cancer.

### BACKGROUND

Natural products (NPs) and their underlying architectures have long been a source of inspiration for developing disease-modulating chemical matter.¹ In particular, bioactive fragment-like NPs provide relevant starting points for hit-to-lead optimization, often exploring regions in chemical space not covered by synthetic small molecules.^{2,3} Despite the pre-validation of NP frameworks its widespread use in chemical biology and molecular medicine is hampered by limited knowledge of both on- and off-targets.^{4,5} Moreover, NPs are rich in substructures often presented by “frequent hitters”,⁶ and the so-called “pan assay interference compounds” (PAINS),^{3,7-9} which may afford intractable assay readouts and attrition in the development of pre-clinical chemical entities. Nonetheless, experimental evidence suggests that NPs provide less promiscuous target engagement profiles when compared to synthetic small molecules.¹⁰

β-Lapachone is a fragment-like, naphthoquinone-based natural product isolated from the lapacho tree (Tabebula avellandedae). β-Lapachone has been shown to display significant antitumor activity upon bioactivation by NAD(P)H:quinone oxireductase 1 (NQO1).¹¹ As such, its prodrug ARQ 761 is currently under clinical trials (clinicaltrials.gov identifiers: NCT02514031 and NCT0150280) for cancerous diseases.

### SUMMARY

The present inventors have found that the β-lapachone (1, FIG. 1) is a potent, ligand efficient and allosteric inhibitor of 5-lipoxygenase (5-LO) and that the modulation of 5-LO activity correlates with anti-cancer activity of β-lapachone.

An aspect of the invention provides a method of treating cancer characterized by 5-lipoxygenase (5-LO) expression comprising administering a β-lapachone compound to a patient in need thereof.

In some embodiments, a cancer may be characterized by 5-lipoxygenase (5-LO) and NAD(P)H:quinone oxireductase 1 (NQO1) expression

A β-lapachone compound may have the formula (A);

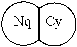

wherein:
   Nq is optionally substituted naphthoquinone:
   Cy is an optionally substituted heterocyclic ring fused to Nq;
and salts, solvates and protected forms thereof.

The β-lapachone compound may have the formula (D):

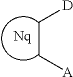

wherein:
   Nq is optionally substituted naphthoquinone;
   A is selected from —OH, —SH, —NH₂ and —NHR, where R is selected from alkyl, aryl and aralkyl;
   D is an optionally substituted alkenyl group,
and salts, solvates and protected forms thereof.

Preferred β-lapachone compounds include β-lapachone and analogues, derivatives and prodrugs thereof, including ARQ 761.

Another aspect of the invention provides a β-lapachone compound for use in a method of treating cancer characterized by 5-lipoxygenase (5-LO) expression.

Another aspect of the invention provides the use of a β-lapachone compound in the manufacture of a medicament for use in a method of treating cancer characterized by 5-lipoxygenase (5-LO) expression.

In another aspect there is provided a β-lapachone compound, such as a compound of formula (A) or (D).

Aspects and embodiments of the invention are described in more detail below.

### BRIEF DESCRIPTION OF FIGURES

FIG. 1 shows the development of REOS/PAINS substructure-containing NPs as potential leads for development. FIG. 1a shows principal component analysis for dimensionality reduction and visualization of fragment-like REOS/PAINS-free chemical entities in ZINC15 (gray) and REOS/PAINS containing fragment-like NPs (red) using RDKit descriptors. FIG. 1b shows box plots of drug-likeness calculated with DataWarrior for FDA-approved drugs, REOS/PAINS-free fragment-like entities in ZINC15, REOS/PAINS-free fragment-like NPs (green) and REOS/PAINS-hitting fragment-like NPs (red). Outliers were excluded. The distribution of drug-likeness was significantly different between FDA-approved drugs and any type of fragment-like NP (two-sided Mann-Whitney test, p<0.0001). Number of molecules analysed: FDA-approved drugs=1506; REOS/PAINS-free fragment-like NPs=35376; REOS/PAINS-hitting fragment-like NPs=35544.

FIG. 2 shows β-lapachone, 1, inhibits human 5-lipoxygenase (5-LO). a) Inhibition of 5-LO in a cell-free assay in presence or absence of 1 mM dithiothreitol (DTT). IC₅₀ (with DTT)=0.24 μM±0.13 log units, n=3. IC₅₀ (without DTT) >30 μM, n=3. Control: zileuton, IC₅₀=1 μM. b) Inhibition of 5-LO by 1 plus 1 mM DTT in presence or absence of 0.01% Triton X100. IC₅₀ (with Triton X100)=0.09 μM±0.21 log units, n=3; IC₅₀ (without Triton X100)=0.12 μM±0.14 log units, n=3. c) Inhibition of 5-LO activity in neutrophil homogenates in presence or absence of 1 mM DTT. IC₅₀ (with DTT)=0.09 μM±0.13 log units, n=3; IC₅₀ (without DTT)=5.2 μM±0.46 log units, n=3. Inhibition by zileuton is independent of DTT.

FIG. 3 shows the biochemical profile of β-lapachone. a) Inhibition of 5-, 12- and 15-LO in intact human neutrophils (without DTT). IC₅₀ (5-LO)=8.6 μM±0.10 log units, n=3; IC₅₀ (12-LO)>30 μM; IC₅₀ (15-LO)>30 μM. b) Inhibition of 5-LO activity in intact human neutrophils supplemented with 1 mM DTT. IC₅₀=0.42 μM±0.11 log units, n=4.

FIG. 4 shows a focused library of β-lapachone-inspired naphthoquinones. FIG. 5a shows structures of the synthesized compounds. FIG. 5b shows screening of the focused library at a concentration of 5 μM against 5-LO (n=2).

FIG. 5 shows a binding model of hydroquinone 1 to 5-lipoxygenase (5-LO) and relationship with the anticancer activity. a) Mutant 5-LO used as template (PDB 3V98). b) Homology model of wild-type human 5-LO. c) Predicted binding pockets with volume>110 Å³. d) Docking pose of hydroquinone 1 into the predicted allosteric pocket. e) Detail of the predicted interactions between 1 and 5-LO.

FIG. 6 shows a competition assay (IC₅₀ curve) between 1 and phosphatidylcholine (PC), n=3. IC₅₀ (3 μg/mL, PC)=100 nM; IC₅₀ (30 μg/mL, PC)=1000 nM. Data advocates for a competition event.

FIG. 7 (left panel) shows 5-LO protein expression. Control: HL-60 cells; Differentiated: DMSO-stimulated HL-60 cells. Centre panel shows IC₅₀ values for 1 against both HL-60 cell line groups. IC₅₀ (differentiated)=0.18 μM; IC₅₀ (control)=0.39 μM, n=1. Right panel shows percentage of live HL-60 cells in the differentiated and control groups when treated with 0.5 μM of 1, n=3. Statistics: two-tailed t-Student test; **p<0.005.

FIG. 8 shows analysis of the NCI-60 panel. FIG. 8a shows the activity distribution of β-lapachone (1, pGI₅₀ z-scores) against the NCI-60 cell lines and grouped by tissue of origin. Outliers were excluded. FIG. 8b shows 5-LO expression across the NCI-60 cancer cell lines. Outliers were excluded. FIG. 8c shows correlation between activity of 1 (pGI₅₀ z-scores) and 5-LO expression in blood cancers (Pearson correlation coefficient=0.85; p=0.03; Confidence interval=95%). FIG. 8d shows correlation in leukemia cell lines (Pearson correlation coefficient=1; p=0.002; Confidence interval=95%). FIG. 8e shows overall survival curves of acute myeloid leukemia (AML) patients with high (red) or low (green) 5-LO expression in two independent cohorts. Higher expression of 5-LO is associated with worst prognosis (p=0.047 in one cohort). Patients were separated by the median of 5-LO expression. Plot generated with PROGgeneV2.⁴¹

FIG. 9 shows in vitro and in vivo assays of β-lapachone, 1, against acute myeloid leukemia (AML) models. FIG. 9a shows concentration-dependent effects of 1 on cell viability. IC₅₀ (HL-60)=2.7 μM±0.04 log units, n=3; IC₅₀ (HEL)=0.9 μM±0.02 log units, n=3. FIG. 9b shows NQO1 and 5-LO protein expression in HL-60 cells. FIG. 9c shows 5-LO rescue experiment by supplementation of 5-HETE lactone (1 μM) and N-acetylcysteine (NAC, 1 μM). Compound 1 was tested at 1 μM. **p<0.01, One-way ANOVA with Tukey HSD post hoc test, n=8. FIG. 9d shows In vivo experiment design. The xenograft model of human AML used relies in the intra-bone marrow injection of 5×10⁵ HEL-GFP cells into the right tibia of previously irradiated (200 rad) NSG mice. Upon tumor trigger in the blood mouse are randomly assigned for treatment with 1 (50 mg/kg intra-peritoneal) or vehicle. FIG. 9e shows survival rates. FIG. 9f shows tumor burden in the blood at day 0 and day 9 after treatment. FIG. 9g shows flow cytometry analysis for tumor infiltration assessment in each indicated organ.

FIG. 10 shows a proposed mechanism of anticancer activity of β-lapachone. β-Lapachone is reduced in situ by NQO1 (or glutathione) to the corresponding hydroquinone form, which inhibits 5-lipoxygenase (5-LO) and elicits cancer cell death. β-Lapachone and its hydroquinone form may present additional drug targets and form reactive oxygen species (ROS) that, together with 5-LO, contribute to the overall phenotypic effects.

FIG. 11 shows IC₅₀ curves for β-lapachone against viability of the leukemia HL-60 cell line.

FIG. 12 shows fluorescence-assisted cell sorting (FACS) data. Intracellular 5-LO was detected in both cell lines. In normal HL-60 cells, the background staining is relatively high and the shift upon 5-LO staining overlaps with the background making it a non-specific staining.

### DETAILED DESCRIPTION

This invention relates to the finding that β-lapachone compounds inhibit 5-lipoxygenase (5-LO) and the inhibition of 5-LO correlates with the cytotoxic effects of the β-lapachone compounds. This finding allows the use of β-lapachone compounds to treat conditions associated with 5-LO expression.

A β-lapachone compound may inhibit 5-LO, and may preferably be a reversible and allosteric antagonist of 5-LO. The β-lapachone compound be a selective inhibitor of 5-LO and/or lipoxygenases.

5-lipoxygenase (5-LO) (also known as arachidonate 5-lipoxygenase, 5LPG; LOG5; 5-LOX) is expressed in bone marrow-derived cells and catalyzes the conversion of arachidonic acid to 5(S)-hydroperoxy-6-trans-8,11,14-cis-eicosatetraenoic acid, and further to the allylic epoxide 5(S)-trans-7,9-trans-11,14-cis-eicosatetrenoic acid (leukotriene A4). 5-LO may be human 5-LO. Human 5-LO (Gene ID 240) has the reference amino acid sequence of NP_000689.1 and may be encoded by the reference nucleotide sequence NM_000698.4.

Techniques for measuring 5-LO activity and assaying inhibitory activity are described elsewhere herein and include cell-free assays (Pufahl et al (2007) Anal Biochem 364 204-212)

The β-lapachone compound for use in the present invention may be an optionally substituted naphthoquinone fused with an optionally substituted heterocyclic ring.

The β-lapachone compound may have the formula (A):

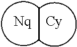

wherein:
Nq is optionally substituted naphthoquinone;
Cy is an optionally substituted heterocyclic ring fused to Nq;
and salts, solvates and protected forms thereof.

The group Nq is optionally substituted naphthoquinone, and preferably Nq is naphthoquinone. The group Nq may be 1,2-naphthoquinone or 1,4-naphthoquinone, and the group Nq is preferably 1,2-naphthoquinone.

Where the naphthoquinone is substituted, the substituents are substituents to the benzene ring of the naphthoquinone. A substituent may be present at one or more, such as one or two, positions on the benzene ring of the naphthoquinone. A substituent may be provided at one or more positions selected from the 5-, 6-, 7- and 8-positions.

The optional substituents for the naphthoquinone, —R^{Np}, may be selected from the group consisting of halo, nitro, cyano, —R^{S1}, —OH, —OR^{S1}, —SH, —SR^{S1}, —NH₂, —NHR^{S1}, —NR^{S1}R^{S2}, —COOH, —COOR^{S1}, —CONH₂, —CONHR^{S1}, —CONR^{S1}R^{S2}, —NHCOR^{S1}, —N(R^{S1})COR^{S1}, where each —R^{S1} and each —R^{S2} is independently alkyl, aryl or aralkyl, which are optionally substituted with halo, or —R^{S1} and —R^{S2} may together form a heterocyclic ring.

Where —R^{S1} or R^{S2} is alkyl, this may be C₁₋₆ alkyl, such as C₁₋₄ alkyl, such as C₁₋₂ alkyl, such as methyl,

Where —R^{S1} or —R^{S2} is aryl, this may be carboaryl, such as C₆₋₁₀ carboaryl, such as phenyl, or heteroaryl, such as C₅₋₁₀ heteroaryl, such as C₅₋₆ heteroaryl.

Where —R^{S1} or —R^{S2} is aralkyl this is an aryl group connected via an alkylene group to the nitrogen atom. The alkylene group may be C₁₋₆ alkylene, such as C₁₋₂ alkylene, such as C₁ alkylene (methylene, —CH₂—). The aryl group may be an aryl group as described above. An example is benzyl.

Where —R^{S1} and —R^{S2} together form a heterocyclic ring, this may be a C₄₋₁₀ heterocyclic ring, such as a C₅₋₆ heterocyclic ring, such as a C₅ heterocyclic ring or a C₆ heterocyclic ring, such as piperidine. The heterocyclic ring may be a single ring or two or more fused rings, where at least one ring is a heterocyclic ring. The heterocyclic ring may contain one or two heteroatoms selected from the group consisting of O, S and N(H).

Cy is preferably fused to the quinone ring of the naphthoquinone group. Where Ar is 1,2-naphthoquinone, Cy is fused to the 3- and 4-positions of the quinone ring in the naphthoquinone group, thus:

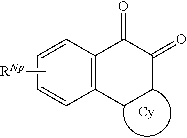

where Cy is an optionally substituted heterocyclic ring, and —R^{Np} represents the one or more optional substituents to the benzene ring of the 1,2-naphthoquinone group.

Where Ar is 1,4-naphthoquinone, Cy is fused to the 2- and 3-positions of the quinone ring, thus:

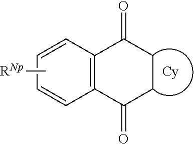

where Cy is an optionally substituted heterocyclic ring, and —R^{Np} represents the one or more optional substituents to the benzene ring of the 1,4-naphthoquinone group.

The group Cy, including the two carbon atoms from the naphthoquinone group to which it is fused, has 5, 6 or 7 ring atoms, such as 5 or 6 ring atoms, such as 5 ring atoms.

Cy is a heterocycle and contains one or two, such as one, ring heteroatoms selected from the group consisting of O, S and N(H). Where there are two ring heteroatoms present, these are not adjacent within the ring.

The ring heteroatom of the heterocycle, such as O, is preferably connected to a carbon ring atom of the quinone ring of the naphthoquinone group.

Where Cy is 1,2-naphthoquinone, the ring heteroatom of the heterocycle may be connected to the carbon ring atom at the 3-position or 4-position, such as the 4-position. Where Cy is 1,4-naphthoquinone, the ring heteroatom of the heterocycle may be connected to the carbon ring atom at the 2-position or 3-position, such as the 2-position.

Cy is preferably an oxygen-containing heterocycle. Thus, a ring atom of the heterocycle is an oxygen atom. The heterocycle may optionally contain one further heteroatom selected from O, S and N(H), though this is less preferred.

The heterocycle is partially unsaturated, by virtue of its fusion to the naphthoquinone group. The heterocycle ring may be fully unsaturated, and it may be aromatic. Preferably, the heterocycle is partially unsaturated.

The group Cy, including the two carbon atoms from the naphthoquinone group to which it is fused, such as the carbon ring atoms from the quinone ring of the naphthoquinone group, may be a 2,3-dihydrofuran group or a 2,5-dihydrofuran group, such as a 2,3-dihydrofuran group.

The group Cy, including the two carbon atoms from the naphthoquinone group to which it is fused, such as the carbon ring atoms from the quinone ring of the naphthoquinone group, may be a 3,4-dihydro-2H-pyran group or a 3,6-dihydro-2H-pyran group, such as a 3,4-dihydro-2H-pyran group.

The heterocyclic ring is optionally substituted, and is preferably substituted. The heterocycle may have one or more substituents, and preferably is mono-, di- or trisubstituted.

A heterocycle carbon ring atom that is a to the ring heteroatom (which ring atom is not also a ring atom of the naphthoquinone group) may be substituted, and is preferably substituted, and is most preferably disubstituted. The α position may be referred to as the 2-position of the heterocycle.

The heterocycle Cy is optionally substituted with one or more substituent groups, —R^{Cy}, selected from the group consisting of —OH, —SH, —NH₂, halo, and -L¹-R⁴, where -L¹- is selected from a covalent bond, alkylene, —O—, —S—, —N(H)—, and —N(R^{L1})—, where —R^{L1} is alkyl, and —R⁴ is selected from the group consisting of alkyl, cycloalkyl, heterocyclyl, and aryl, where each of the alkyl, cycloalkyl, heterocyclyl, and aryl is independently optionally substituted, such as independently optionally substituted with one or more groups —R^{T} as described below. The group -L¹- is not alkylene when —R⁴ is alkyl.

The heterocycle may be substituted with one, two, or three groups —R^{Cy}. Preferably, the heterocycle is substituted with three groups —R^{Cy}. Most preferably two of those three groups are alkyl, such as methyl. Where there are two or more groups —R^{Cy}, two of these groups may be connected to the same heterocycle ring carbon atom (geminal substitution).

The heterocycle may be substituted, such as disubstituted, with alkyl, and optionally further substituted with —OH.

The heterocycle may be substituted, such as disubstituted, with alkyl, and optionally further substituted with -L¹-R⁴.

In one embodiment, -L¹- is selected from a covalent bond, alkylene, —O— or —N(H).

The group -L¹- is preferably selected from —N(H)—, alkylene or a covalent bond.

The group -L¹- may be a covalent bond. The group -L¹- may be a covalent bond. The group -L¹- may be —N(H)—.

Where -L¹- is an alkylene group this may be C₁₋₆ alkylene, such as C₁₋₂ alkylene, such as C₁ alkylene (methylene, —CH₂—).

The group —R^{L1} is alkyl, such as C₁₋₆ alkyl, such as C₁₋₂ alkyl, such as methyl.

The group —R⁴ is preferably aryl, such as carboaryl or heteroaryl. The aryl group is optionally substituted.

A carboaryl group may be phenyl or naphthyl, such as phenyl.

A heteroaryl group may C₅₋₁₀ heteroaryl such as C₅₋₆ heteroaryl, such as C₅ heteroaryl. Examples of C₅ heteroaryl are triazolyl, such as 1,2,3-triazolyl, furanyl, thiophenyl, pyrrolyl, and oxazolyl, and examples of C₆ heteroaryl are pyridinyl and pyrimidinyl.

The aryl group may be selected from phenyl and C₅ heteroaryl, such as triazolyl, such as 1,2,3-triazoyl.

Where —R⁴ is alkyl, the may be C₁₋₆ alkyl, such as C₁₋₄ alkyl, such as C₁₋₂ alkyl, such as methyl.

Where —R⁴ is alkenyl, this may be C₂₋₆ alkenyl, such as C₂₋₄alkenyl, such as C₂₋₃ alkenyl, such as C₂ alkenyl (vinyl) or C₃ alkenyl (allyl).

Where —R⁴ is alkynyl, this may be C₂₋₆ alkynyl, such as C₂₋₄ alkynyl, such as C₂₋₃ alkynyl, such as C₃ alkynyl (propargyl).

The alkyl, alkenyl and alkynyl groups may be linear or branched.

Where —R⁴ is cycloalkyl, this may be C₃₋₁₀ cycloalkyl, such as C₅₋₆ cycloalkyl, such as C₆ cycloalkyl (cyclohexyl). The cycloalkyl may be a single ring or two or more fused rings.

Where —R⁴ is heterocyclyl, this may be C₄₋₁₀ heterocyclyl, such as C₅₋₆ heterocyclyl, such as C₅ heterocyclyl or C₆ heterocyclyl, such as piperidinyl. The heterocyclyl may be a single ring or two or more fused rings, where at least one ring is a heterocyclyl ring. The heterocyclyl may contain one or two heteroatoms selected from the group consisting of O, S and N(H).

Where —R⁴ is alkyl, cycloalkyl, heterocyclyl, or aryl, these groups may be optionally substituted by one or more groups —R^{T}, where each —R^{T} is independently selected from —OH, —SH, —NH₂, halo, and -L²-L³-R⁵ where -L²- is selected from a covalent bond, —O—, —S—, —N(H)—, —N(R^{L2})—, where —R^{L2} is alkyl, and —C(O)—, -L³- is selected from a covalent bond, alkylene and alkenylene, and —R⁵ is selected from the group consisting of alkyl, alkenyl, cycloalkyl, heterocyclyl, aryl and a saccharide group, where each of the alkyl, alkenyl cycloalkyl, heterocyclyl, aryl and the saccharide group is independently substituted. The group -L³- is a covalent bond when —R⁵ is alkyl or alkenyl.

Where —R⁴ is alkyl, cycloalkyl, heterocyclyl, or aryl, these groups may be optionally substituted by one group -R^{T}.

The group —R^{T} is preferably halo or -L²-L³-R⁵, such as -L²-L³-R⁵.

The group -G³- is preferably a covalent bond. Here, the oxygen heterocycle is a 5-membered heterocyclic ring (dihydrofuran ring).

The group -G³-may be alkylene, such as C₁₋₂ alkylene, such as —CH₂— or —CH₂CH₂—, such as —CH₂—. When -G³- is alkylene, —CH₂— is preferred. Here, the oxygen heterocycle is a 6-membered heterocyclic ring (dihydropyran ring). Where, -G³- is —CH₂CH₂—, the oxygen heterocycle is a 7-membered heterocyclic ring.

The group -L²- is preferably a covalent bond or —C(O)—.

The group -L³- is preferably selected from alkylene and alkenylene, Where -L³- is alkylene this may be C₁₋₆ alkylene, such as C₁₋₂ alkylene, such as C₁ alkylene (methylene, —CH₂—).

Where -L³- is alkenylene this may be C₂₋₆ alkenylene, such as C₂₋₃ alkenylene, such as C₂ alkenylene, such as —CH═CH—.

Where —R⁵ is alkyl, this may be C₁₋₆ alkyl, such as C₁₋₄ alkyl, such as C₁₋₂ alkyl, such as methyl.

Where —R⁵ is alkenyl, this may be C₂₋₆ alkenyl, such as C₂₋₄alkenyl, such as C₂₋₃ alkenyl, such as C₂ alkenyl (vinyl) or C₃ alkenyl (allyl).

Where —R⁵ is alkynyl, this may be C₂₋₆ alkynyl, such as C₂₋₄alkynyl, such as C₂₋₃ alkynyl, such as C₃ alkynyl (propargyl).

The alkyl, alkenyl and alkynyl groups may be linear or branched.

Where —R⁵ is cycloalkyl, this may be C₃₋₁₀ cycloalkyl, such as C₅₋₆ cycloalkyl, such as C₆ cycloalkyl (cyclohexyl). The cycloalkyl may be a single ring or two or more fused rings.

Where —R⁵ is heterocyclyl, this may be C₄₋₁₀ heterocyclyl, such as C₅₋₆ heterocyclyl, such as C₅ heterocyclyl or C₆ heterocyclyl, such as piperidinyl. The heterocyclyl may be a single ring or two or more fused rings, where at least one ring is a heterocyclyl ring. The heterocyclyl may contain one or two heteroatoms selected from the group consisting of O, S and N(H).

Where —R⁵ is aryl, this may be carboaryl, such as C₆₋₁₀ carboaryl, such as phenyl, or heteroaryl, such as C₅₋₁₀ heteroaryl, such as C₅₋₆ heteroaryl.

Where —R⁵ is a saccharide group, this may be a monosaccharide. A monosaccharide may be a triose, tetrose, pentose or hexose, and is preferably a hexose.

The monosaccharide may be an aldose or a ketose, and is preferably an aldose.

The monosaccharide may be glucose.

The saccharide group may be connected, and is preferably connected, via an oxygen atom, such as an oxygen atom of a hydroxyl group, of a saccharide, which may be the hydroxyl group in the hemiacetal group of the cyclic form of the saccharide group (typically the hydroxyl group attached to the carbon atom at the 1-position).

An amino sugar, such as glucosamine, may be regarded as a saccharide in the present case. In some embodiments the saccharide may be a carbohydrate having only oxygen, carbon and hydrogen.

Where —R⁵ is alkyl, alkenyl, cycloalkyl, heterocyclyl, or aryl, these groups may be optionally substituted by one or more groups selected from the group consisting of —OH, —OR^{Q}, —SH, —SR^{T}, —NH₂, —NHR^{Q}, —N(R^{Q})₂, halo, nitro, —COOH, —C(O)OR^{Q}, —OC(O)R^{Q}, —CONH₂, —C(O)NHR^{Q}, —C(O)N(R^{Q})₂ and —NHC(O)R^{Q}, where each —R^{Q} is independently alkyl or aralkyl.

The group —R^{Q} may be C₁₋₆ alkyl, such as methyl or ethyl, such as methyl.

The group —R^{Q} may be benzyl.

Where —R⁵ is a saccharide group, the hydroxyl functionality may be acylated (—C(O)Me). One or more, such as all, the groups may be acylated. Similarly, where amino functionality is present, for example in an amino sugar, that functionality may be acylated.

A ring atom of the heterocycle may be unsubstituted, mono-substituted or di-substituted.

The 2-position of the heterocycle fused to the naphthoquinone may be mono- or disubstituted, and each substituent may be alkyl, such as C₁₋₆ alkyl, such as methyl. Preferably, the 2-position is disubstituted with methyl.

A heterocycle carbon ring atom that is p to the ring heteroatom (which ring atom is not also a ring atom of the naphthoquinone group) may be substituted, and may be monosubstituted.

Where the heterocycle contains more than 5 ring atoms, a heterocycle carbon ring atom that is γ to the ring heteroatom (which ring atom is not also a ring atom of the naphthoquinone group) may be substituted, and is preferably substituted, and is most monosubstituted.

Where the heterocycle contains a nitrogen ring atom, N(H), this may be optionally substituted a group selected from alkyl, aryl and aralkyl.

In a preferred embodiment, the compound of formula (A) is a compound (B):

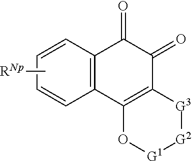

wherein:
-G¹- is —CH₂—, —CHR¹— or —C(R¹)(R²)—,
-G²- is —CH₂— or —CHR³—;
-G³- is a covalent bond or alkylene;
where each of —R¹, —R² and —R³ is independently a group —R^{Cy}, such as a group -L¹-R⁴,
and salts, solvates and protected forms thereof.

Each group —R^{Cy} is as defined above.

The group -G¹- is most preferably —C(R¹)(R²)—. Here, each of R¹ and R² is independently selected from -L¹-R⁴.

Preferably each of —R¹ and —R² is alkyl, such as C₁₋₆ alkyl, such as C₁₋₂ alkyl, such as C₁ alkyl (methyl).

The group -G¹- is preferably —C(CH₃)₂—.

The group —R¹ may be the same as —R².

The group -G²- may be —CH₂—.

The group -G²- may be —CHR³—.

The compound of formula (A) is preferably β-lapachone:

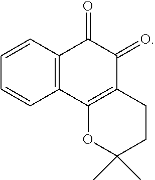 [SMILES: CC1(C)CCC2=C(O1)c1ccccc1C(=O)C2=O]

The compound of formula (A) may be a compound (C):

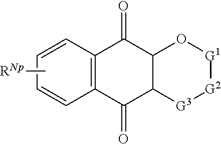

wherein —R^{Np}, -G¹-, -G²- and -G³- have the same meanings as the compounds of formula (C).

The β-lapachone compound may be β-lapachone (3,4-dihydro-2,2-dimethyl-2H-naphtho[1,2-b]pyran-5,6-dione) or an analogue, derivative or prodrug of β-lapachone. β-lapachone (CAS 4707-32-8) may be obtained from commercial suppliers, synthesised using standard techniques or isolated from the lapacho tree (Tabebuia avellanedae) in accordance with standard methods.

The invention also provides the use of a β-lapachone compound of formula (D):

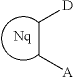

wherein:
   Nq is optionally substituted naphthoquinone;
   -A is selected from —OH, —SH, —NH₂ and —NHR, where R is selected from alkyl, aryl and aralkyl;
   -D is an optionally substituted alkenyl group,
and salts, solvates and protected forms thereof.

A compound of formula (D) may be referred to as a Lapachol compound.

The groups -A and -D are preferably substituents to the quinone ring of the optionally substituted naphthoquinone. The groups -A and -D are located on neighbouring ring atoms, such as neighbouring ring atoms of the quinone ring of the optionally substituted naphthoquinone.

The group -A is preferably selected from —OH, —NH₂ and —NHR, more preferably from —OH and —NH₂, and most preferably -A is —OH.

The group -Nq may be a group as described above for the compounds of formula (A). For example, Nq may be optionally substituted naphthoquinone, and preferably Nq is naphthoquinone. The group Nq may be 1,2-naphthoquinone or 1,4-naphthoquinone or, and the group Nq is preferably 1,2-naphthoquinone.

Where —R is alkyl, this may be C₁₋₆ alkyl, such as C₁₋₄ alkyl, such as C₁₋₂ alkyl, such as methyl.

Where —R is aryl, this may be carboaryl, such as C₆₋₁₀ carboaryl, such as phenyl, or heteroaryl, such as C₅₋₁₀ heteroaryl, such as C₅₋₆ heteroaryl.

Where —R is aralkyl this is an aryl group connected via an alkylene group to the nitrogen atom. The alkylene group may be C₁₋₆ alkylene, such as C₁₋₂ alkylene, such as C₁ alkylene (methylene, —CH₂—).

The aryl group may be an aryl group as described above. An example is benzyl.

The group —R may be a group —R^{L1} as defined above.

The alkenyl double bond in -D is preferably not conjugated with the quinone double bond.

The group -D is preferably:

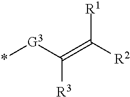

where —R¹, —R², —R³ and -G³- have the same meanings as —R¹, —R², —R³ and -G³- for the compounds of formula (B).

The compound of formula (D) is preferably Lapachol:

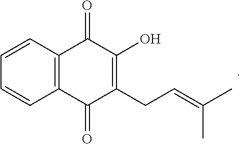 [SMILES: CC(C)=CCC1=C(O)C(=O)c2ccccc2C1=O]

The compounds of the invention may be provided as solvates, for example as hydrates.

The compounds of the invention also include the salts forms of the compounds of formula (A), (B), (C) and (D) mentioned above, where appropriate, for example where the compounds possess appropriate functional groups, such as carboxyl groups (—COOH) and amine groups (such as —NH₂). These salts may be pharmaceutically acceptable salts.

The compounds of the invention may also be provided in a protected form, including a prodrug form.

For example, one or both carbonyl groups of the β-lapachone compound may be protected as an imine or an acetal, and these groups may be removed prior to use in the methods of treatment described herein, or they may be administered in the imine or acetal form, with the expected cleavage of these groups in vivo. WO 2012/040513 describes suitable imine and acetal forms for protection of the carbonyl groups of β-lapachone compounds.

In one embodiment, the β-lapachone compound is provided in an imine protected form. The imine group may protect one or both, such as one, of the carbonyl groups of the β-lapachone compound, such as the carbonyl group at the 1-position.

An optionally substituted phenylimine group may be provided in place of a carbonyl group in the β-lapachone. The phenyl group may be unsubstituted or substituted with one or more groups a selected from alkyl, such as methyl, alkoxy, such as methoxy, nitro and halo, such as bromo.

Typically the phenyl group is unsubstituted or monosubstituted, and the monosubstituent may be provided at the 4-position.

For example, the β-lapachone compound may be ARQ 761.

Where the compound of the invention possesses a hydroxyl group, such as may be present in the compound of formula (D), this hydroxyl group may be protected, for example as an ester, such as an alkyl ester, or a silyl ether.

The compounds for use in the invention may be obtained from commercial suppliers, may be extracted from natural materials, or may be prepared by synthesis, using starting materials that are commercially available or extracted from natural materials. Derivatives of β-Lapachone, for example derivatives having a modified and/or substituted heterocycle, are well known in the art.

For example, Lapachol may be isolated from the heartwood of Tabebuia sp. β-Lapachone may be prepared directly from Lapachol. Other compounds of the invention may be prepared via nor-lapahcol, or a derivatised version of this compound. For example, as described in the worked examples herein, nor-lapahcol may be derivatised by bromonation of the double bond, followed by reaction with a suitable nucleophile, such as an oxygen, sulfur or nitrogen nucleophile, H—R^{Cy} (for example when —R^{Cy} is -L¹-R⁴ and -L¹- is —O—, —S—, —N(H)— or —N(R^{L1})—, and —R4 is as previously described).

A patient suitable for treatment as described herein may have a disease that is characterized by 5-LO expression. In some embodiments, the disease may be further characterized by expression of NQO1.

NQO1 (NAD(P)H quinone dehydrogenase; also known as DTD; QR1; DHQU; DIA4; NMOR1; or NMORI) is a cytoplasmic 2-electron reductase that reduces quinones to hydroquinones and has been associated with cancer susceptibility. NQO1 may be human NQO1. Human NQO1 (Gene ID 1728) may have the reference amino acid sequence of NP_000894.1 and may be encoded by the reference nucleotide sequence NM_000903.2.

Diseases characterized by 5-LO expression may include cancer and inflammatory diseases.

Inflammatory diseases suitable for treatment as described herein may include allergy, asthma, acne vulgaris, autoimmune diseases, coeliac disease, chronic prostatitis, colitis, inflammatory bowel disease, cystitis, mastocytosis, rheumatoid arthritis, hepatitis, preperfusion injury, glomerulonephritis, lupus erythematosus, rhinitis, transplant rejection and vasculitis. Other suitable inflammatory diseases are disclosed in Sharma et al (2006) Inflammopharmacology 14 (1-2) 10-16.

Cancer suitable for treatment as described herein may be any type of solid or non-solid cancer or malignant lymphoma and especially leukaemia, sarcomas, skin cancer, bladder cancer, breast cancer, uterine cancer, ovarian cancer, prostate cancer, lung cancer, colorectal cancer, cervical cancer, liver cancer, head and neck cancer, oesophageal cancer, pancreatic cancer, renal cancer, stomach cancer and cerebral cancer. Cancers may be familial or sporadic. The cancer may be a metastatic cancer. In some preferred embodiments, the cancer may be kidney, colon or blood cancer.

In preferred embodiments, the cancer may be a blood cancer, for example, a leukaemia such as CML, AML, CLL or ALL, most preferably AML.

Cancer characterized by 5-LO expression may comprise one or more cancer cells in the patient that have increased expression of 5-LO relative to control cells. For example, the expression of 5-LO in one or more cancer cells in the patient may be greater than the expression in a control cell or greater than a predetermined threshold value.

Cancer characterized by 5-LO and NQO1 expression may comprise one or more cancer cells in the patient that have increased expression of both 5-LO and NQO1 relative to control cells. For example, the expression of 5-LO and NQO1 in one or more cancer cells in the patient may be greater than the expression in a control cell or greater than a predetermined threshold value.

A cancer characterized by 5-LO expression and optionally NQO1 expression may be identified by any suitable technique. For example, the expression of 5-LO in one or more cancer cells from the patient may be determined. Suitable techniques for determining the expression of target genes in a cell are well-known in the art and include fluorescent activated cell sorting (FACS), PCR-based methods, such as reverse transcription PCR (RT-PCR), quantitative RT-PCR (qPCR), TaqMan™, or TaqMan™ low density array (TLDA), microarrays, multi-analyte profile testing, radioimmunoassay (RIA), northern blot assay, Western blot assay, immunofluorescent assay, enzyme immunoassay, enzyme linked immunosorbent assay (ELISA), immunoprecipitation assay, chemiluminescent assay, immunohistochemical assay, dot blot assay, or slot blot assay or proteomics-based methods.

The patient may have been previously identified as having a disease characterized by 5-LO expression or be at risk of having or at risk of a disease characterized by 5-LO expression.

A method may comprise identifying the patient as having or at risk of a disease characterized by 5-LO expression before administration.

An individual suitable for treatment as described above may be a mammal, such as a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orang-utan, gibbon), or a human.

In some preferred embodiments, the individual is a human. In other preferred embodiments, non-human mammals, especially mammals that are conventionally used as models for demonstrating therapeutic efficacy in humans (e.g. murine, primate, porcine, canine, or leporid) may be employed.

In some embodiments, the individual may have minimal residual disease (MRD) after an initial cancer treatment.

An individual with a cancer characterized by 5-LO expression may display at least one identifiable sign, symptom, or laboratory finding that is sufficient to make a diagnosis of cancer in accordance with clinical standards known in the art. Examples of such clinical standards can be found in textbooks of medicine such as Harrison's Principles of Internal Medicine, 15th Ed., Fauci A S et al., eds., McGraw-Hill, New York, 2001. In some instances, a diagnosis of a cancer in an individual may include identification of a particular cell type (e.g. a cancer cell) in a sample of a body fluid or tissue obtained from the individual. In some embodiments, the individual may have been previously identified or diagnosed with cancer characterized by 5-LO expression or a method of the invention may comprise identifying or diagnosing cancer characterized by 5-LO expression in the individual for example by determining the presence of an identifiable sign, symptom, or laboratory finding indicative of cancer characterized by 5-LO expression in the individual.

While it is possible for a β-lapachone compound, such as β-lapachone, to be administered to the individual alone, it is preferable to present the compound in a pharmaceutical composition or formulation.

A pharmaceutical composition may comprise, in addition to the β-lapachone compound, one or more pharmaceutically acceptable carriers, adjuvants, excipients, diluents, fillers, buffers, stabilisers, preservatives, lubricants, or other materials well-known to those skilled in the art. Such materials should be non-toxic and should not interfere with the efficacy of the active compound. The precise nature of the carrier or other material will depend on the route of administration, which may be by bolus, infusion, injection or any other suitable route, as discussed below. Suitable materials will be sterile and pyrogen free, with a suitable isotonicity and stability. Examples include sterile saline (e.g. 0.9% NaCl), water, dextrose, glycerol, ethanol or the like or combinations thereof. The composition may further contain auxiliary substances such as cyclodextrins, wetting agents, emulsifying agents, pH buffering agents or the like.

Suitable carriers, excipients, etc. can be found in standard pharmaceutical texts, for example, Remington's Pharmaceutical Sciences, 18th edition, Mack Publishing Company, Easton, Pa., 1990.

The term “pharmaceutically acceptable” as used herein pertains to compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

The formulations may conveniently be presented in unit dosage form and may be prepared by any methods well-known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

The β-lapachone compound or pharmaceutical compositions comprising the β-lapachone compound may be administered to a subject by any convenient route of administration, whether systemically/peripherally or at the site of desired action, including but not limited to, oral (e.g. by ingestion); and parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly. Usually administration will be by the oral route, although other routes such as intraperitoneal, subcutaneous, transdermal, intravenous, nasal, intramuscular or other convenient routes are not excluded.

The pharmaceutical compositions comprising the β-lapachone compounds may be formulated in a dosage unit formulation that is appropriate for the intended route of administration.

Formulations suitable for oral administration (e.g. by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or moulding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g. sodium lauryl sulfate); and preservatives (e.g. methyl p-hydroxybenzoate, propyl p-hydroxybenzoate, ascorbic acid). Moulded tablets may be made by moulding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for parenteral administration (e.g. by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example, from about 10 ng/ml to about 1 g/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

Optionally, other therapeutic or prophylactic agents may be included in the pharmaceutical composition or formulation.

β-lapachone compounds as described herein may be useful in the treatment of cancers characterized by 5-LO expression and optionally NQO1 expression.

Treatment may be any treatment or therapy, whether of a human or an animal (e.g. in veterinary applications), in which some desired therapeutic effect is achieved, for example, the inhibition or delay of the onset or progress of the condition, and includes a reduction in the rate of progress, a halt in the rate of progress, inhibition of metastasis, amelioration of the condition, cure or remission (whether partial or total) of the condition, preventing, delaying, abating or arresting one or more symptoms and/or signs of the condition or prolonging survival of a subject or individual beyond that expected in the absence of treatment.

Cancer growth generally refers to any one of a number of indices that indicate change within the cancer to a more developed form. Thus, indices for measuring an inhibition of cancer growth include a decrease in cancer cell survival, a decrease in tumor volume or morphology (for example, as determined using computed tomographic (CT), sonography, or other imaging method), a delayed tumor growth, a destruction of tumor vasculature, improved performance in delayed hypersensitivity skin test, an increase in the activity of cytolytic T-lymphocytes, and a decrease in levels of tumor-specific antigens. Inhibition of 5-LO in an individual with cancer, as described herein may improve the capacity of the individual to resist cancer growth, in particular growth of a cancer already present the subject and/or decrease the propensity for cancer growth in the individual.

Treatment as described herein may include prophylactic treatment (i.e. prophylaxis) i.e. the individual being treated may not have or may not be diagnosed as having a cancer characterised by 5-LO expression at the time of treatment. For example, an individual susceptible to or at risk of the occurrence or re-occurrence of a cancer characterised by 5-LO expression may be treated as described herein. Such treatment may prevent or delay the occurrence or re-occurrence of the cancer characterised by 5-LO expression in the individual or reduce its symptoms or severity after occurrence or re-occurrence. In some embodiments, the individual may have been previously identified as having increased susceptibility or risk of cancer characterised by 5-LO expression compared to the general population or a method may comprise identifying an individual who has increased susceptibility or risk of cancer characterised by 5-LO expression. Prophylactic or preventative treatment may be preferred in some embodiments.

β-lapachone compounds may be administered as described herein in therapeutically-effective amounts.

The term “therapeutically-effective amount” as used herein, pertains to that amount of an active compound, or a combination, material, composition or dosage form comprising an active compound, which is effective for producing some desired therapeutic effect, commensurate with a reasonable benefit/risk ratio.

The appropriate dosage of β-lapachone compounds may vary from individual to individual. Determining the optimal dosage will generally involve the balancing of the level of therapeutic benefit against any risk or deleterious side effects of the administration. The selected dosage level will depend on a variety of factors including, but not limited to, the route of administration, the time of administration, the rate of excretion of the active compound, other drugs, compounds, and/or materials used in combination, and the age, sex, weight, condition, general health, and prior medical history of the individual. The amount of active compounds and route of administration will ultimately be at the discretion of the physician, although generally the dosage will be to achieve therapeutic plasma concentrations of the active compound without causing substantial harmful or deleterious side-effects.

In general, a suitable dose of the active compound is in the range of about 100 μg to about 400 mg per kilogram body weight of the subject per day, preferably 200 μg to about 200 mg per kilogram body weight of the subject per day. Where the active compound is a salt, an ester, prodrug, or the like, the amount administered is calculated on the basis of the parent compound and so the actual weight to be used is increased proportionately. For example, 50 to 100 mg of β-lapachone compound may be orally administered twice daily in capsule or tablet form.

A β-lapachone compound may be orally administered in an amount sufficient to maintain the serum concentration at a level that yields>50% inhibition of 5-LO.

Administration in vivo can be effected in one dose, continuously or intermittently (e.g., in divided doses at appropriate intervals).

Methods of determining the most effective means and dosage of administration are well known in the art and will vary with the formulation used for therapy, the purpose of the therapy, the target cell being treated, and the subject being treated. Single or multiple administrations can be carried out with the dose level and pattern being selected by the physician.

Multiple doses of the β-lapachone compound may be administered, for example 2, 3, 4, 5 or more than 5 doses may be administered. The administration of the β-lapachone compound may continue for sustained periods of time. For example treatment with β-lapachone compound may be continued for at least 1 week, at least 2 weeks, at least 3 weeks, at least 1 month or at least 2 months.

Treatment with the β-lapachone compound may be continued for as long as is necessary to reduce cancer symptoms or achieve complete remission.

The β-lapachone compound may be administered alone or in combination with other treatments, either simultaneously or sequentially dependent upon the individual circumstances. For example, a β-lapachone compound as described herein may be administered in combination with one or more additional active compounds.

The β-lapachone compound may be administered in combination with a second therapeutic agent.

The second therapeutic agent may be an anti-cancer compound, for example, an anti-cancer compound is selected from an anthracycline, such as daunorubicin or idarubicin, cytarabine, vincristine, mitoxantrone, L-asparaginase, cyclophosphamide, fibromun, dacarbazine, methotrexate, 6-mercaptopurine, chlorambucil, an alkylating agent, decitabine, azacitidine, cyclophosphamide, corticosteroids, imatinib, midostaurin, cladribine, pentostatin, fludarabine, topotecan, rituximab, chlorambucil, a taxane, such as paclitaxel, and doxorubicin. In some preferred embodiments, the second therapeutic compound may be gemcitabine or nab-paclitaxel.

The β-lapachone compound may be administered in combination with irradiation. The use of irradiation for the treatment of cancer conditions is well known in the art.

The expression of 5-LO may also be useful in identifying cancers that are that are likely to be responsive (“sensitive”) or non-responsive (“resistant”) to treatment with a β-lapachone compound and to selecting patients suitable for treatment with a R-lapachone compound. A method of selecting a cancer patient for treatment with a β-lapachone compound comprising
providing a sample of cancer cells from a cancer patient, and
determining the presence or amount of 5-LO expression in the cancer cells.

A cancer patient with cancer cells that express 5-LO; express 5-LO at a level above a threshold value or at higher levels than a control cell may be selected for treatment with the β-lapachone compound. Techniques suitable for determining the presence or amount of expression of 5-LO in cells are well-known in the art.

A patient may have a cancer characterized by 5-LO expression as described above.

The present inventors have also identified a correlation between 5-LO expression and poor prognosis in cancer patients (e.g. a survival rate of less than 50% over 3 years), in particular patients with blood cancers such as CLL and AML.

Another aspect of the invention provides a method of prognosis of a cancer patient comprising
determining the amount of 5-LO expression in cancer cells obtained from a cancer patient,
the level of 5-LO expression being indicative of the prognosis of the patient.

The patient may have a cancer as described above, for example a blood cancer, such as CLL or AML.

The amount of expression of 5-LO in the cancer cells may be determined using any suitable technique.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Certain aspects and embodiments of the invention will now be illustrated by way of example and with reference to the figures described above.

### Experiments

Materials

Reagents and solvents were purchased from ABCR Chemicals, Sigma Aldrich, Alfa Aesar, Acros, Fluka or TCI Deutschland and used without further purification. Proton and carbon nuclear magnetic resonance (¹H and ¹³C NMR) spectra were recorded on a Bruker AVANCE DRX400 MHz and Varian Mercury 500 MHz spectrometers. All chemical shifts are quoted on the δ scale in ppm using with TMS as an internal reference. Coupling constants (J) are reported in Hz with the following splitting abbreviations: s=singlet, d=doublet, t=triplet, dd=doublet of doublets, td=triplet of doublets, m=multiplet. All compounds present ≥95% purity unless otherwise stated.

Lapachol

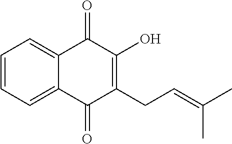 [SMILES: CC(C)=CCC1=C(O)C(=O)c2ccccc2C1=O]

Lapachol (2-hydroxy-3-(3′-methyl-2′-butenyl)-1,4-naphthoquinone) was extracted from the heartwood of Tabebuia sp. (Tecoma). Initially, a saturated aqueous sodium carbonate solution was prepared and added to the sawdust of ipe tree. Upon observing rapid formation of lapachol sodium salt (deep red coloration), hydrochloric acid was added, allowing the precipitation of lapachol. Then, the solution was filtered and a yellow solid was obtained. This solid was purified by a series of recrystallizations with hexane as a solvent. Lapachol was then obtained as crystalline yellow solid with high purity (>99%).

¹H NMR (400 MHz, CDCl₃, 303 K) δ: 8.11 (dd, 1H, J=7.6 and 1.4 Hz), 8.06 (dd, 1H, J=7.6 and 1.4 Hz), 7.74 (td, 1H, J=7.6 and 1.4 Hz), 7.66 (td, 1H, J=7.6 and 1.4 Hz), 7.36 (s, 1H), 5.25-5.17 (m, 1H), 3.31 (d, 2H, J=7.3 Hz), 1.79 (s, 3H), 1.69 (s, 3H). ¹³C NMR (100 MHz, CDCl₃, 303 K) δ: 184.4, 181.5, 152.5, 134.7, 133.7, 132.7, 129.3, 126.6, 125.9, 123.3, 119.5, 25.6, 22.5, 17.8.

### β-Lapachone (1)

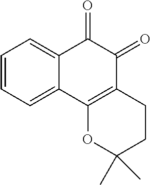 [SMILES: CC1(C)CCC2=C(O1)c1ccccc1C(=O)C2=O]

Sulfuric acid was slowly added to lapachol (1 mmol, 242 mg) until complete dissolution of the quinone and formation of a red solution. Then, the solution was poured into ice and the precipitate formed was filtered off and washed with water. β-Lapachone was recrystallized in ethanol and obtained as an orange solid (240 mg, 99% yield); mp 153-155° C. ¹H NMR (400 MHz, CDCl₃, 303 K) δ: 8.06 (dd, 1H, J=7.6 and 1.4 Hz), 7.81 (dd, 1H, J=7.8 and 1.1 Hz), 7.65 (ddd, 1H, J=7.8, 7.6 and 1.4 Hz), 7.51 (td, 1H, J=7.6, 7.6 and 1.1 Hz), 2.57 (t, 2H, J=6.7 Hz), 1.86 (t, 2H, J=6.7 Hz), 1.47 (s, 6H). ¹³C NMR (100 MHz, CDCl₃, 303 K) δ: 179.8, 178.5, 162.0, 134.7, 132.6, 130.6, 130.1, 128.5, 124.0, 112.7, 79.3, 31.6, 26.8, 16.2. The data are consistent with those reported in the literature (see 42 and 43).

### Nor-Lapachol

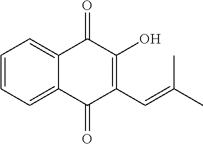 [SMILES: CC(C)=CC1=C(O)C(=O)c2ccccc2C1=O]

Nor-lapachol was synthesized by the Hooker oxidation and data are consistent with those reported in the literature (see 48 to 50). In a 500 mL flask was added 4.84 g of lapachol and then 40 mL THF. Separately a solution of 2.4 g of anhydrous Na₂CO₃ in 50 mL of H₂O was prepared, which was also added to the flask, forming a dark red solution. Under heating, 60° C., about 6.0 mL of 30% H₂O₂ in about 1 hour, 10 in 10 drops, was slowly added until total solution discolouration to form a pinkish color solution. The solution was made from 0° C. to −2° C. Concentrated HCl was then added dropwise under stirring until a white precipitate appeared. The mixture was left in the refrigerator for 2 hours and then vacuum filtered, yielding a white solid, obtained in 70% yield. This substance was dissolved in 32 mL of THF. Then a solution of 1.43 g of Na₂CO₃ in 59.25 mL of H₂O was added. A solution left under mechanical stirring for the precipitate to dissolve. To the solution was added 19.75 mL of 25% NaOH, followed by another solution of CuSO₄.5H₂O, 11.85 g in 59.25 ml of H₂O. under constant stirring. The solution was given in a water bath for 1 hour and 45 minutes, then filtered on Celite® (infusion ground) to give a filtered wine color. The solution was acidified with concentrated HCl to form an orange precipitate, which was filtered and washed successively with distilled H₂O until complete neutralization. Nor-lapachol was obtained as an orange solid (160 mg, 0.7 mmol, 70% yield); mp 121-122° C.^{37 1}H NMR (400 MHz, CDCl₃, 303 K) δ: 8.13 (ddd, 1H, J=7.5, 1.5 and 0.5 Hz), 8.10 (ddd, 1H, J=7.5, 1.5 and 0.5 Hz), 7.76 (td, 1H, J=7.5, 7.5 and 1.5 Hz), 7.69 (td, 1H, J=7.5, 7.5 and 1.5 Hz), 6.03-5.99 (m, 1H), 2.0 (d, 3H, J=1.5 Hz), 1.68 (d, 3H, J=1.2 Hz). ¹³C NMR (100 MHz, CDCl₃, 303 K) δ: 184.7, 181.5, 151.1, 143.6, 134.9, 133.0, 132.9, 129.5, 126.9, 126.0, 120.9, 113.6, 26.5, 21.7.

### 3-Arylamino-nor-β-lapachone

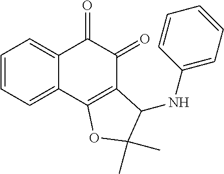 [SMILES: CC1(C)OC2=C(C(=O)C(=O)c3ccccc32)C1Nc1ccccc1]

Nor-lapachol (228 mg, 1.0 mmol) was dissolved in 25 mL of dichloromethane, followed by the addition of 2 mL of bromine. A bromo intermediate precipitated immediately as an orange solid. Dicholoromethane was added and the solvent evaporated in vacuum to remove of bromine. An excess of the required aniline (2 mmol) was added and the mixture was stirred overnight. The crude reaction mixture was poured into 50 mL of water. The organic phase was separated and washed with 10% HCl (3×50 mL), dried over sodium sulfate, filtered, and evaporated under reduced pressure to yield a solid, which was purified by column chromatography in silica gel and eluted with an increasing polarity gradient mixture of hexane and ethyl acetate (9/1 to 7/3).^{41,42} 3-Arylamino-nor-β-lapachone was obtained as a red solid (303 mg, 95% yield); mp 126-128° C. ¹H NMR (400 MHz, CDCl₃, 303 K) δ: 8.00 (d, 1H, J=7.4 Hz), 7.62 (dt, 1H, J=14.8 and 7.4 Hz), 7.54 (dd, 1H, J=10.5 and 4.2 Hz), 7.10 (t, 1H, J=7.8 Hz), 6.66 (t, 1H, J=7.3 Hz), 6.50 (d, 1H, J=7.9 Hz), 4.72 (s, 1H), 3.87 (sl, 1H), 1.59 (s, 3H), 1.50 (s, 3H). ¹³C NMR (100 MHz, CDCl₃, 303 K) δ: 181.17, 175.62, 169.77, 147.58, 134.84, 132.74, 131.42, 129.74, 129.56, 127.69, 125.29, 118.35, 115.47, 113.36, 97.09, 61.92, 27.52, 22.00. Data are consistent with those reported in the literature (see 44).

### p-Chloro-3-arylamino-nor-β-lapachone

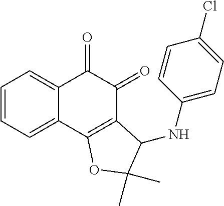 [SMILES: CC1(C)OC2=C(C(=O)C(=O)c3ccccc32)C1Nc1ccc(Cl)cc1]

p-Chloro-3-arylamino-nor-β-lapachone synthesized as above and obtained as a red solid (317 mg, 0.9 mmol, 90% yield); mp 210-214° C.). ¹H NMR (400 MHz, CDCl₃) δ: 8.1 (ddd, 1H, J=7.9, 2.2 and 0.7 Hz), 7.72-7.60 (m, 3H), 7.13 (dd, 2H, J=6.7 and 2.1 Hz), 6.5 (dd, 2H, J=6.7 and 2.1 Hz), 4.75 (d, 1H, J=5.7 Hz), 1.66 (s, 3H), 1.56 (s, 3H); ¹³C NMR (100 MHz, CDCl₃) δ: 180.8, 175.3, 169.6, 145.8, 134.6, 132.6, 131.1, 129.5, 129.1, 127.2, 125.0, 122.6, 114.6, 114.1, 96.6, 61.7, 27.3, 21.7.

### 3-Hydroxy-nor-β-lapachone

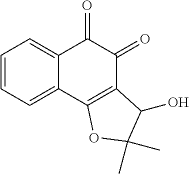 [SMILES: CC1(C)OC2=C(C(=O)C(=O)c3ccccc32)C1O]

A solution of nor-lapachol (228 mg, 1.0 mmol) in 25 mL of dichloromethane, and 2 mL of bromine was prepared. The bromo intermediate precipitated immediately as an orange solid. The reaction mixture was transferred to the separatory funnel and extracted with sodium bisulfite (3×10 mL) to form the hydroxylated product. The product was obtained as an orange solid (170 mg, 70% yield); mp 110-112° C. 1H NMR (400 MHz, CDCl₃, 303 K) δ: 7.96 (dd, 1H, J=7.5 and 0.7 Hz), 7.58 (dtd, 2H, J=8.8, 7.5 and 1.3 Hz), 7.51 (td, 1H, J=7.4 and 1.6 Hz); 4.95 (s, 1H), 3.82 (s, 1H), 1.56 (s, 3H), 1.40 (s, 3H). ¹³C NMR (100 MHz, CDCl₃, 303 K) δ: 181.5, 176.4, 171.2, 134.8, 132.8, 131.4, 129.6, 127.7, 125.4, 117.8, 97.2, 75.3, 26.8, 20.8. Data are consistent with those reported in the literature (see 45).

### 3-Hydroxy-β-lapachone

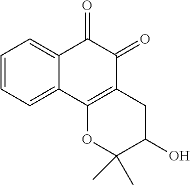 [SMILES: CC1(C)OC2=C(CC1O)C(=O)C(=O)c1ccccc12]

To a solution of lapachol (100 mg, 0.42 mmol) in dichloromethane (20 mL) was added m-chloroperbenzoic acid (90 mg, 0.52 mmol) and the mixture was stirred at room temperature for 24 h. The reaction mixture was washed with a saturated solution of sodium bicarbonate, water, dried over magnesium sulfate, and the solvent was evaporated. The residue was purified by column chromatography on silica gel using chloroform/ethyl acetate (2:1) as eluent to afford the product. The product was obtained as a red solid (54 mg, 50% yield); mp 202-204° C. ¹H NMR (400 MHz, CDCl₃, 303 K) δ: 7.98 (dd, 1H, J=7.6 and 0.9 Hz), 7.74 (d, 1H, J=7.8 Hz), 7.60 (td, 1H, J=7.7 and 1.3 Hz), 7.46 (td, 1H, J=7.6 and 1.0 Hz), 4.18 (dd, 1H, J=7.3 and 5.4 Hz), 3.14 (dd, 1H, J=18.1 and 5.4 Hz), 2.91 (dd, 1H, J=18.1 and 7.4 Hz), 1.56 (s, 3H), 1.53 (s, 3H). ¹³C NMR (100 MHz, CDCl₃, 303 K) δ: 179.3, 178.2, 161.3, 135.2, 131.9, 131.4, 130.3, 129.1, 124.5, 111.3, 81.3, 50.2, 28.2, 26.4, 23.9. Data are consistent with those reported in the literature (see 46 and 47).

### 3-((4-Cinnamoylphenyl)amino)-2,2-dimethyl-2,3-dihydronaphtho[1,2-b]furan-4,5-dione

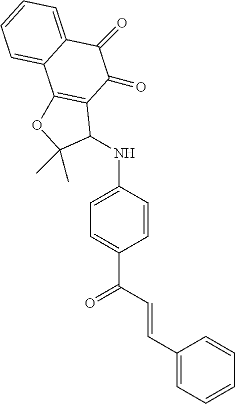 [SMILES: CC1(C)OC2=C(C(=O)C(=O)c3ccccc32)C1Nc1ccc(C(=O)/C=C/c2ccccc2)cc1]

The required chalcone was prepared by condensing 4′-aminoacetophenone with the respective aldehyde in the presence of sodium hydroxide in ethanol. Then, an excess of bromine (2 mL) was added to a cooled solution of nor-lapachol (228 mg, 1 mmol) in 25 mL of dichloromethane. The brominated intermediate was obtained as an orange solid. After the removal of excess bromine, a solution of the respective chalcone (1 mmol) in 25 mL of dichloromethane was added and stirred overnight. The reaction mixture was concentrated under reduced pressure and the residue was purified by column chromatography in silica gel by eluting with an increasing polarity gradient mixture of hexane and ethyl acetate.⁴⁷ The title compound was obtained as a red solid. (157 mg, 0.35 mmol, 35% yield); mp 196-199° C., ¹H NMR (500 MHz, CDCl₃) δ: 7.98 (d, 1H, J=7.4 Hz), 7.88 (d, 2H, J=8.4 Hz), 7.69 (dt, 3H, J=14.8 and 11.6 Hz), 7.59 (t, 3H, J=7.4 Hz), 7.49 (d, 1H, J=15.6 Hz), 7.39-7.35 (m, 3H), 6.61 (d, 2H, J=8.7 Hz), 4.91 (d, 1H, J=7.3 Hz), 1.69 (s, 3H), 1.57 (s, 3H). ¹³C-APT NMR (125 MHz, CDCl₃) δ: 187.8, 180.7, 175.2, 170.0, 151.3, 142.9, 135.3, 134.6, 132.7, 131.1, 131.0, 129.9, 129.5, 128.8, 128.2, 128.0, 127.2, 125.2, 121.9, 114.5, 112.2, 96.7, 60.8, 27.4, 21.8.

### (2S,3S,4R,5S,6S)-2-(Acetoxymethyl)-6-((1-(2,2-dimethyl-4,5-dioxo-2,3,4,5-tetrahydronaphtho[1,2-b]furan-3-yl)-1H-1,2,3-triazol-4-yl)methoxy)tetrahydro-2H-pyran-3,4,5-triyl triacetate

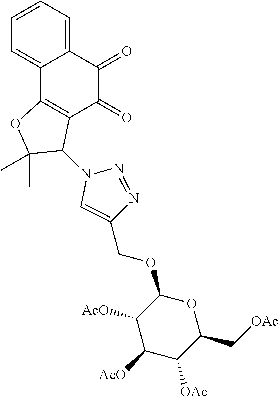 [SMILES: CC(=O)OC[C@@H]1O[C@H](OCc2cn(C3C4=C(OC3(C)C)c3ccccc3C(=O)C4=O)nn2)[C@@H](OC(C)=O)[C@H](OC(C)=O)[C@H]1OC(C)=O]

To a mixture of 3-azido-nor-β-lapachone (269 mg, 1.0 mmol) with CuSO₄.5H₂O (12.48 mg, 5 mol %) and sodium ascorbate (19.81 mg, 5 mol %) in 8 mL CH₂Cl₂/H₂O (1:1 v/v), the required alkyne-carbohydrate (1.1 equivalents) was added. The mixture was stirred overnight at room temperature. The organic phase was extracted with dichloromethane, dried with NaSO₄ and concentrated under reduced pressure. The residue obtained was purified by column chromatography on silica gel using as eluent a gradient mixture of hexane/ethyl acetate with increasing polarity. The nor-β-lapachone-based 1,2,3-triazole-carbohydrate was obtained as a yellow solid (537 mg, 0.82 mmol, 82% yield); mp 88-90° C. ¹H NMR (400 MHz, CDCl₃) δ: 8.20-8.17 (m, 1H), 7.82-7.71 (m, 3H), 7.55/7.51 (s, 1H), 5.98/5.97 (s, 1H), 5.25-5.15 (m, 1H), 5.07 (t, 1H, J=10 Hz), 5.00-4.78 (m, 3H), 4.67 (t, 1H, J=7.6 Hz), 4.30-4.10 (m, 2H), 3.77-3.70 (m, 1H), 2.07, 2.01, 2.00, 1.98, 1.97, 1.77 (six singlets observed, 12H), 1.19 (s, 3H), 0.98-0.83 (m, 3H). ¹³C NMR (100 MHz, CDCl₃) δ: 180.0, 180.0, 174.6, 174.5, 171.3, 171.2, 170.7, 170.6, 170.1, 170.1, 169.6, 169.5, 169.4, 144.4, 144.2, 134.8, 134.8, 133.4, 133.4, 131.6, 131.5, 130.0, 130.0, 126.6, 126.6, 125.6, 125.5, 122.6, 122.3, 111.1, 100.2, 99.9, 95.9, 95.8, 72.8, 72.6, 71.9, 71.3, 71.2, 68.3, 68.3, 67.0, 66.9, 63.3, 63.0, 61.8, 61.7, 31.5, 29.0, 27.7, 27.6, 22.6, 22.6, 21.1, 20.7, 20.6, 20.5, 20.5, 20.4, 14.1, 11.4.

### (2S,3S,4R,5S,6S)-2-(acetoxymethyl)-6-((1-((2,2-dimethyl-4,5-dioxo-2,3,4,5-tetrahydronaphtho[1,2-b]furan-3-yl)methyl)-1H-1,2,3-triazol-4-yl)methoxy)tetrahydro-2H-pyran-3,4,5-triyl triacetate

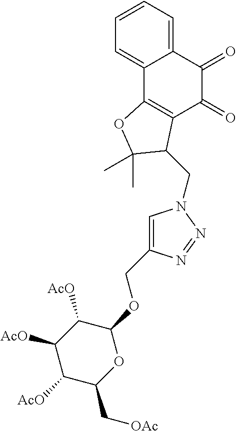 [SMILES: CC(=O)OC[C@@H]1O[C@H](OCc2cn(CC3C4=C(OC3(C)C)c3ccccc3C(=O)C4=O)nn2)[C@@H](OC(C)=O)[C@H](OC(C)=O)[C@H]1OC(C)=O]

To a mixture of 256 mg (1 mmol) of the required azide quinone with CuSO₄.5H₂O (12.48 mg, 5 mol %) and sodium ascorbate (19.81 mg, 5 mol %) in 8 mL CH₂Cl₂/H₂O (1:1 v/v), the corresponding alkyne-carbohydrate (1.1 equivalents) was added. The mixture was stirred overnight at room temperature. The organic phase was extracted with dichloromethane, dried over NaSO₄ and concentrated under reduced pressure. The obtained residue was purified by column chromatography on silica gel using as eluent a gradient mixture of hexane/ethyl acetate with increasing polarity.⁴⁸ The quinone-based 1,2,3-triazole-carbohydrate was obtained as a yellow solid (544 mg, 0.8 mmol, 85% yield); mp 117-119° C. 1H NMR (400 MHz, CDCl₃) δ: 8.06 (bs, 1H), 7.76-7.67 (m, 2H), 7.61-7.59 (bs, 2H), 5.53-5.48 (m, 1H), 5.25-4.70 (m, 8H), 4.25-4.15 (m, 2H), 3.77 (d, 1H, J=4 Hz), 3.38-3.32 (m, 1H), 2.98-2.93 (m, 1H), 2.08 (s, 3H), 2.04 (s, 3H), 2.01 (s, 3H), 1.97 (s, 3H). ¹³C NMR (100 MHz, CDCl₃) δ: 180.4, 178.7, 175.2, 173.8, 170.6, 170.1, 169.4, 144.7, 134.9, 132.4, 130.5, 129.7, 126.8, 124.6, 123.8, 114.7, 100.0, 84.5, 72.7, 72.0, 71.2, 68.3, 63.2, 61.8, 53.4, 29.7, 20.7, 20.7, 20.6, 20.6.

Methods

1. Database Analyses

A natural product collection comprising 515,581 structures from the Universal Natural Product Database, ZINC database and Traditional Chinese Medicines database was assembled. The library was standardized with the “wash” function in MOE 2015.10 (Chemical Computing Group, Canada) and duplicates were filtered out to afford 428,308 unique natural products. Fragment-like entities in ZINC15 and FDA-approved drugs were collected from ZINC15 and DrugBank v5.0, respectively, and processed identically. PAINS and REOS filters were employed as implemented in Canvas (Schrödinger LLC) to afford 342,114 fragment-like entities from ZINC15, 36,365 fragment-like PAINS/REOS-free natural products, and 37.942 fragment-like PAINS/REOS-containing natural products. Murcko scaffolds, Morgan fingerprints (radius 2, 2048 bits), RDKit and CATS descriptors were calculated in KNIME through RDKit and MOE native nodes. Principal component analyses were performed in KNIME. Drug-likeness was computed with DataWarrior.⁵⁷ Data was plotted with Python 2.7.10.

2. Target Prediction

Target prediction was carried out on the publically available SPiDER web server (ETH Zurich), as previously reported.⁵⁸⁻⁶² In short, β-lapachone and lapachol were projected onto self-organizing maps together with reference compounds from the COBRA database.⁶³ Chemical structures are processed with the “wash” function of the Molecular Operating Environment (MOE, Chemical Computing Group, Montreal, Canada), prior to description with the CATS2⁶⁴ and MOE2D descriptors. Predictions are carried out by calculating the Euclidean distances of the molecules to the reference compounds in COBRA. The output comprises target families at a confidence level of p<5%. The distances are converted to p values, according to a pre-calculated background distribution of distances between molecules annotated to bind different targets. The arithmetic average of these p values serves as confidence score for the target prediction. With the background distribution of confidence scores, each prediction can be associated with another p value that indicates the statistical significance of the prediction.⁵⁸ SEA⁶⁵ and SuperPred⁶⁶ predictions were performed from the respective web servers.

Ligand and bioactivity data for targets of interest was collected from ChEMBL22. Ligands were normalized with the “wash” function in MOE 2015.10 and bioaffinity data (Kᵢ, K_{D}, IC₅₀ or EC₅₀) transformed to the respective antilog value (pAffinity). Regression random forest models were built for each individual target using CATS2 descriptors. For each target, 500 models were built without tree depth and a minimum split node size of 2. The models were subjected to 10-fold cross validation and mean average errors calculated.

3. Binding Pocket Prediction

A homology model of 5-lipoxygenase (5-LO) was constructed with Swiss-Model (https://swissmodel.expasy.org), by reinstating the native enzyme sequence into a mutant apo (PDB 3V98⁶⁷) 5-LO structure. Hydrogen atoms, charges and energy minimization (Amber10:EHT force field) was performed with MOE 2015.10 (Chemical Computing Group, Montreal, Canada), as well as binding pocket prediction. Only pockets with a volume >110 Å³ were considered for further study.⁶⁸ The steroechemical quality of the model was assessed through Ramachandran plots.

4. Molecular Docking

The structure of β-lapachone was “washed” and energy minimized with MOE2015.10, prior to docking into the predicted binding pockets with GOLD 5.4.1. Docking runs were performed with the apo 5-LO model. Default settings, including the scoring function ChemPLP were used.⁶⁹ Several docking runs using different pocket centres were performed. Five hundred genetic algorithm runs were performed, and the top 10% poses were saved for manual inspection.

5. Dynamic Light Scattering

Dynamic light scattering (Zetasizer Nano S, Malvern, UK) was used to determine compound colloidal aggregation potential and kinetic solubility. The particle sizes were measured at 25° C. Water solubility was measured as described elsewhere with successive measurements within 60 minutes.⁵⁶ A 100 mM stock solution of β-lapachone was prepared in DMSO, following dilution to deionized water to obtain an analyte solution of 100 μM (0.1% DMSO). Colloidal aggregation was measured through sequential dilutions. Solubility was assessed at 25 μM after 0 and 30 minutes.

6. Intrinsic Tryptophan Fluorescence

5-Lipoxygenase (Cat No. ab114310, Abcam) was concentrated to 0.5 μM in buffer (50 mM Tris-HCl, pH 8.0, 5 mM CaCl₂). A stock solution of β-lapachone was prepared in DMSO and added to the enzyme solution in a concentration range of 0-10 μM. The final DMSO concentration was kept under 0.1%. Fluorescent measurements were performed using a 1 cm pathlength quartz cuvette. Spectra were collected in an Edinburgh Instruments FLS920 Series Fluorescence Spectrophotometer at 25° C. with fluorescence excitation and scanning emission set to 295 nm and 310 to 450 nm, respectively. All assays were carried out in triplicate.

7. Expression, Purification and Cell-Free Activity Assay of Human Recombinant 5-LO.

E. coli(BL21) was transformed with pT3-5-LO plasmid, and recombinant 5-LO protein was expressed at 30° C. as described.³⁴ Cells were lysed in 50 mM triethanolamine/HCl pH 8.0, 5 mM EDTA, 1 mM phenylmethanesulphonyl fluoride, soybean trypsin inhibitor (60 μg/mL), and lysozyme (1 mg/mL), homogenized by sonication (3×15 s), and centrifuged at 40,000×g for 20 min at 4° C. The 40,000×g supernatant (S40) was applied to an ATP-agarose column to partially purify 5-LO as described.⁷⁰ Aliquots of semi-purified 5-LO (0.5 μg) were diluted with 1 mL ice-cold PBS containing 1 mM EDTA. Samples were pre-incubated with the test compound or vehicle (0.1% DMSO) with or without Triton X-100 and/or 1 mM DTT, as indicated. After 10 min at 4° C., samples were pre-warmed for 30 s at 37° C., and 2 mM CaCl₂ plus the indicated concentrations of arachidonic acid (AA) were added to start the formation of 5-LO products. After 10 min, the reaction was stopped by addition of one volume of ice-cold methanol, and the formed 5-LO products were analyzed by RP-HPLC as described.⁷¹ 5-LO products include the all-trans isomers of LTB₄ (tr-LTB₄ isomers) as well as 5(S)-hydroperoxy-6-trans-8,11,14-cis-eicosatetraenoic acid (5-HPETE) and its corresponding alcohol 5(S)-hydroxy-6-trans-8,11,14-cis-eicosatetraenoic acid (5-HETE). For phosphatidylcholine competition assays (3 or 30 μg/ml PC) samples were pre-incubated on ice with β-lapachone with or without 1 mM DDT or vehicle (0.1% DMSO) for 10 minutes. After addition of 2 mM CaCl₂) and 20 μM AA the incubation was continued for 10 minutes at 37° C. and after that stopped with methanol.

8. Neutrophil Isolation

Peripheral blood (University Hospital Jena, Germany) was withdrawn from healthy adult volunteers with consent that had not taken any anti-inflammatory drugs during the last 10 days, by venipuncture in heparinized tubes (16 IE heparin/mL blood). The blood was centrifuged at 4000 g for 20 min at 20° C. for preparation of leukocyte concentrates. Leukocyte concentrates were then subjected to dextran sedimentation and centrifugation on lymphocyte separation medium (LSM 1077, PAA, Colbe, Germany). Contaminating erythrocytes of pelleted neutrophils were removed by hypotonic Iysis. Neutrophils were then washed twice in ice-cold PBS and finally resuspended in PBS pH 7.4 containing 1 mg/mL glucose or in PBS pH 7.4 containing 1 mg/mL glucose plus 1 mM CaCl₂ (PGC buffer) (purity>96-97%).

Method A: 5-Lipoxygenase (5-LO), 12-lipoxygenase (12-LO) and 15-lipoxygenase-2 (15-LO-2) inhibition assays were performed at Cerep, SA (Celle l'Evescault, France) on a fee-for-service basis (Ref 0772³⁶, 0883³⁷ and 0893³⁸, respectively), as described in Table 1.

TABLE 1
Lipoxygenase assay protocols.

| | | Measured | | Detection |
|---|---|---|---|---|
| Assay | Substrate | component | Incubation | method |
| 5-LO | Arachidonic acid (25 | Rhodamine 123 | 20 min/rt | Fluorimetry |
| | μM) | | | |
| 12-LO | Arachidonic acid (4 | Ferric oxidation of | 5 min/rt | Photometry |
| | μM) | xylenol orange | | |
| 15-LO-2 | Arachidonic acid (10 | 15S-HpETE | 90 min/30 | Fluorometry |
| | μM) | | min | |

Method B. For determination of 5-LO products in intact neutrophils, the cells (5×10⁶) were resuspended in 1 mL PGC buffer, preincubated for 15 min at 37° C. with β-lapachone or vehicle (0.1% DMSO), and incubated for 10 min at 37° C. with 2.5 μM Ca²⁺-ionophore A23187 plus 20 μM AA. The reaction was stopped on ice by addition of 1 mL of methanol, 30 μL 1 N HCL, 500 μL PBS, and 200 ng prostaglandin B₁ were added, and the samples were subjected to solid phase extraction on C18-columns (100 mg, UCT, Bristol, Pa., USA). 5-LO products (LTB₄, tr-LTB₄ isomers, and 5-HETE), were analyzed by RP-HPLC and quantities calculated on the basis of the internal standard PGB₁. Cysteinyl-LTs C₄, D₄ and E₄ were not detected (amounts were below detection limit), and oxidation products of LTB₄ were not determined.

For analysis of 5-LO product formation in corresponding homogenates, neutrophils were resuspended in PBS containing 1 mM EDTA for 5 min at 4° C. and sonicated (4×10 s, 4° C.). Homogenates, corresponding to 5×10⁶ cells/mL, were incubated with the test compounds or vehicle (0.1% DMSO) with or without 1 mM DTT for 15 min at 4° C., pre-warmed for 30 s at 37° C., and the reaction was started by the addition of 2 mM CaCl₂ plus the indicated concentrations of AA (routinely 20 μM). The reaction was stopped after 10 min and the samples were analyzed as described for intact cells above.

Data are expressed as mean±S.E.M. IC₅₀ values were calculated from averaged measurements at 6 different concentrations of the compound by nonlinear regression using GraphPad Prism software (San Diego, Calif.) one site binding competition. Statistical evaluation of the data was performed by one-way ANOVA followed by a Tukey-Kramer post-hoc test for multiple comparisons respectively. A p value<0.05 (*) was considered significant.

9. Phosphodiesterase 5 Assay

Phosphodiesterase 5 (PDE5) assay was performed at Cerep, SA (Celle l'Evescault, France) on a fee-for-service basis (Ref 0204³⁹), as described in Table 2.

TABLE 2
Phosphodiesterase assay protocol.

| | | Measured | | Detection |
|---|---|---|---|---|
| Assay | Substrate | component | Incubation | method |
| Inhibition | [³H]cGMP + cGMP | [³H]5′GMP | 60 min/rt | Scintillation |
| | (1 μM) | | | counting |

10. Minimum Inhibitory Concentration (MIC) Assays

Escherichia coli K12 and Staphylococcus aureus ATCC 25923 were grown overnight at 37° C. and re-inoculated in 24-well plates containing 2.5 mL of Luria Bertani medium (LB) to give an optical density of ˜0.01 at 600 nm. Stock solutions of β-lapachone, and lapachol were prepared in DMSO (1% final concentration) and added to the cell suspensions to obtain final concentrations between 5-210 μM. Wells containing a growth control (cell suspensions with 1% DMSO) and a sterile media control were also prepared. The plates were incubated for 18 h at 37° C. and 90 r.p.m. The concentration of compound in the first well in the series that presented no sign of visible growth was reported as the MIC. The OD600 of the cultures was also measured. All the MIC wells were serially diluted in phosphate buffer saline (PBS) and plated onto LB agar plates. Growth was evaluated after 24 h of incubation at 37° C. to access the minimum bactericidal concentration (MBC).

11. Cancer Cell Assays

HL-60 cells were routinely cultured in RPMI medium supplemented with 10% FBS, 1% Pen-strep and 1% HEPES at 5×10⁵ cells/mL. 5-LO overexpression is stimulated by initially starving the cells in medium with 1% FBS for at-least 2-3 passages followed by growing the cells in 1.5% DMSO for another 3-4 days.⁵⁵ For intracellular staining of 5-LO, 10⁶ cells were collected and concentrated in 50 mL PBS. Cells were fixed with 100 mL BD fix buffer while vortexing. Cells were thoroughly washed with PBS and permeabilized using Perm Buffer followed by one hour incubation with primary anti 5-LO antibody (AB376, Merck Millipore). After the required incubation time, cells were washed and incubated for another hour with goat anti-rabbit Alexa fluor 488 antibody. Cells are then washed and resuspended in PBS and acquired by LSRFortessa™ flow cytometer (BD Biosciences, USA) with a 488 nm laser, a 505 nm long-pass filter and a 530/30 nm band-pass filter (for FITC detection). Data was analyzed using the FlowJo software.

HL-60 cells with and without DMSO stimulation were seeded at a concentration of 5×10⁵ cells/mL in a 96 well plate format. Cells were treated with varying concentration of β-lapachone for a time period of 48 hours. Cell death was analyzed using standard Alamar Blue assay. Data is represented after being normalized to the vehicle control.

Results

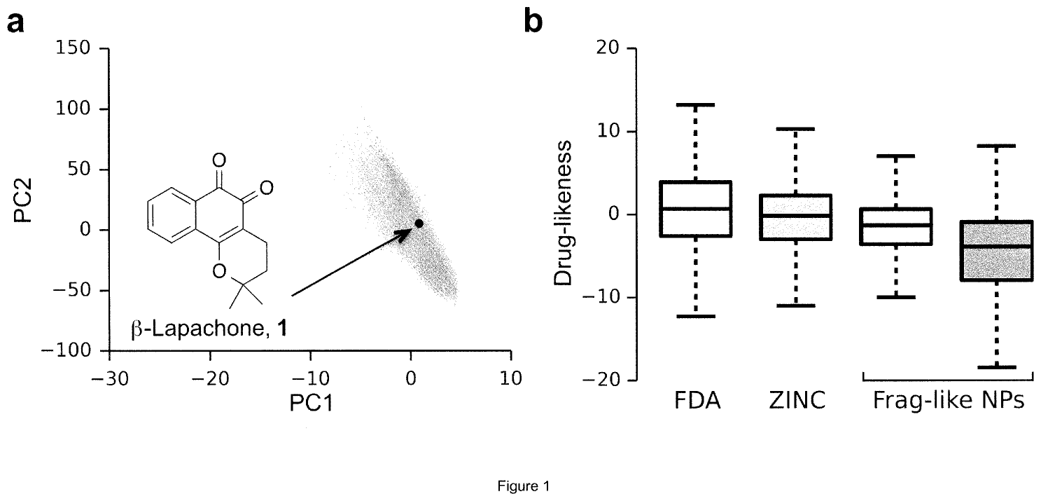

We set out to clarify the polypharmacological profile of 1 (β-lapachone) and probe the underlying molecular basis for macromolecular target recognition. Interestingly, our structural analysis of the chemical matter assayed by Clemons et al.¹⁰ shows that only 30% of NPs pass the rapid elimination of swill (REOS)¹³ and PAINS filters, whereas 53% of the more promiscuity-prone synthetic molecules are both REOS and PAINS substructures-free. Moreover, from a substructural and topological pharmacophore level, we found overlapping chemical spaces between REOS/PAINS-complying and violating chemical matter (FIG. 1a). On the other hand, drug-likeness of FDA-approved drugs, “clean” fragments in ZINC15 and REOS/PAINS-hitting fragment-like NPs, including 1, is highly distributed, suggesting it as a poor metric for prioritizing small molecules for screening campaigns (FIG. 1b). Thus, our data shows that we cannot rationalize the exclusion of structurally “ugly” compounds from screening libraries as a general approach. Of note, we computed that only ca. 50% of FDA-approved drugs are fully compliant with these filters, suggesting that proper hit validation rather than a priori exclusion of chemical entities from screening campaigns may be more appropriate in select cases to avoid missed opportunities.

With that in mind, we used a ligand-based machine learning algorithm^{14,15} to confidently predict potential macromolecular counterparts for 1 based on a consensus of physicochemical and topological pharmacophore features. In opposition to other tools using substructural fingerprints,^{16,17} a range of targets covering different families were predicted with the SPiDER webserver, supporting the engagement of multiple targets to mediate both pharmacological and toxicological profiles of 1. Importantly, known targets for 1 (e.g. DNA topoisomerase,¹⁸ cyclooxygenase¹⁹) were predicted retrospectively, advocating for the appropriateness of our approach. From an algorithm point of view, competing tools likely underperformed in this case due to the array of descriptors used. In fact, 1 presents a scaffold that matches structures mostly annotated to phenotype assay readouts without factual knowledge of target engagement, further supporting the lack of confident predictions by competing tools.

With confident predictions in hand, we prioritized 228 kinases, 5-LO, and selected G-protein coupled receptors (GPCRs) and transient receptor potential channels for additional analyses, since they had also been confidently predicted for lapachol—an isomer of 1—and downstream assays were readily accessible. We sought additional prediction confidence by building the Drug-Target Relationship Predictor (DEcRyPT) machine intelligence workflow that uses regression random forest technology as an orthogonal learning approach to self-organizing maps. In doing so, we aimed at curating confident predictions from SPiDER by stimating an affinity value for 1 against the 236 pre-selected targets.

DEcRyPT was built using manually and automatically curated ChEMBL22 data to solely include relevant bioactivity information for model assembly. The bioactivity annotations were normalized by transforming the affinity data into a −log₁₀ value (pAffinity). The CATS2 topological pharmacophore descriptors were then calculated for each reference ligand (MOE, CCG Canada implementation).⁵¹ In short, CATS2 combinatorially autocorrelates pharmacophore features within a molecule (positive/negative charge, lipophilic, aromatic, hydrogen bond donor/acceptor) up to a topological distance of 10 bonds. Given the ‘fuzziness’ of the molecular descriptor, the resulting high dimensional vector can be used to leverage machine intelligence in the absence of apparent ligand structure similarity, which is arguably ideal for both de novo designed compounds⁵² and NPs³. The built models were subjected to stratified 10-fold cross-validation to assess quality. An average mean absolute error of 0.533±0.103 log units suggests the general utility of the computed models. Moreover, the models support a range of different activities for 1, providing complementary information to clustering algorithms, such as SPiDER, and further allowing screening prioritizations.

We screened 1 against targets from distinct families, including 4 kinases, 4 G-protein coupled receptors (Prostanoid EP₁₋₄), 2 transient receptor potential channels (TRPV1 and TRPM8), and 1 enzyme (5-lipoxygenase, 5-LO), using functional assays. We prioritized these targets as all of them had also been confidently predicted for lapachol (2)—a structural isomer of 1—and due to assay accessibility. At a concentration of 150 μM, i.e. where 50% effect equates to a ligand efficiency (LE) of ca. 0.30, we found that 1 interfered with the kinase assay technology and presented only weak TRP channel effects. Activation of EP receptors was only relevant in the case of subtype EP₄ (53% activation by 1). On the other hand, both 1 and 2 showed antagonist effects against EP₂ (60% effect by 1), EP₃ (95% and 50% effects by 1 and 2, respectively) and EP₄ (56% effect by 2). The TRPV1 channel was equally antagonized by 1 and 2 (63% and 65% effect, respectively), whereas TRPM8 was only antagonized by 2 (91% effect). Gratifyingly, both compounds potently inhibited 5-LO at the assayed concentration (96% and 87% for 1 and 2, respectively). The observed range of activities against unrelated targets suggests specific target recognition, which is also advocated by diverging target engagement profiles of the two isomers in some cases.

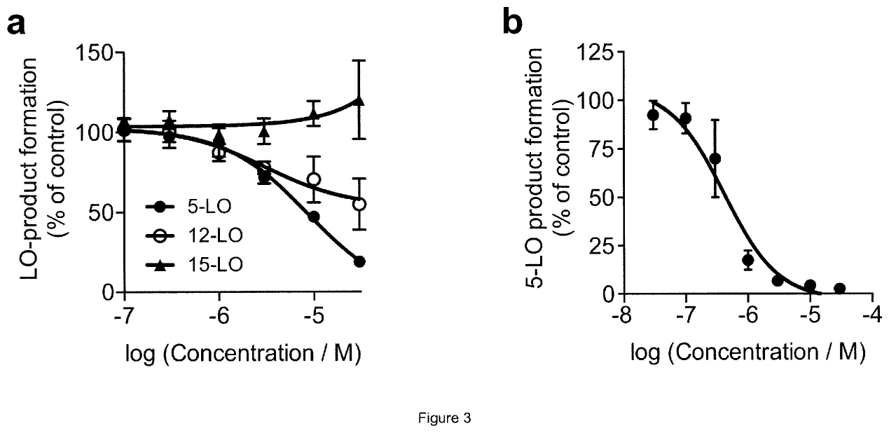

Motivated by the preliminary screening data, we endeavored to obtain concentration-response curves against targets that had shown >85% activity modulation, i.e. EP₃/5-LO for 1 and TRPM8/5-LO for 2. The compounds displayed concentration-dependent effects against the assayed targets and good ligand efficiencies (LE>0.30), supporting that more potent entities inspired in these scaffolds may be obtained upon medicinal chemistry optimization. Among these, 1 potently inhibited 5-LO (IC₅₀=2.1 μM±0.23 log units; LE=0.44; FIG. 3a) in a cell-free assay and in reducing conditions. An orthogonal 5-LO functional assay corroborated the obtained data. Albeit more modest, 1 inhibited EP₃ with potency identical to that predicted by DEcRyPT. Our data thus show the accuracy and general utility of the novel method for the prediction of target affinities. Using circular dichroism as orthogonal technology, we confirmed binding of 1 to 5-LO and determined the protein melting curves at different ligand concentrations. A K_{D} value of 5.7 μM was measured for 1. Significantly, no colloidal aggregation that could potentially lead to artefactual 5-LO inhibition was measured through dynamic light scattering at relevant concentrations.

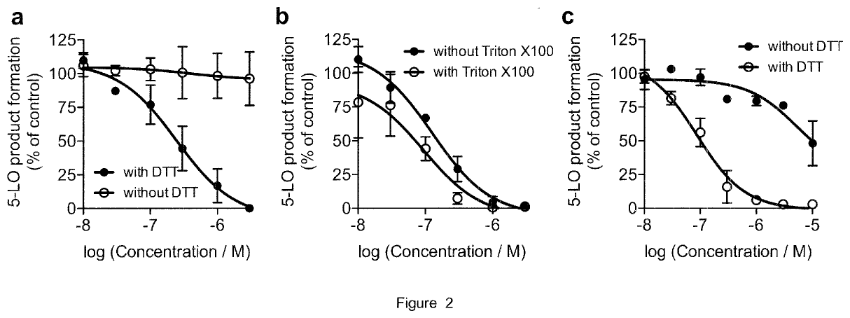

To understand the molecular basis of 5-LO inhibition by 1, we conducted the cell-free functional assay in the absence of dithiothreitol, which led to negligible 5-LO modulation up to a concentration of 30 μM (FIG. 2a). From a learning algorithm point of view, the result is puzzling as the drug target predictions were carried out with ortho-quinone 1. However, it is possible that the active species are ill-annotated for a fraction of quinone/ortho-quinone 5-LO inhibitors included in ChEMBL22 and that were used as training dataset; this can justify the prediction outcome. The presence or absence of 0.01% Triton X100 also led to identical concentration-response curves in cell-free 5-LO inhibition assays (FIG. 2b), which again rules out unspecific interference by 1. We then analysed 5-LO inhibition in intact human neutrophils stimulated with A23187 and exogenous arachidonic acid to assess the efficiency of 1 as a leukotriene biosynthesis inhibitor.¹⁹ β-Lapachone suppressed 5-LO product formation with an IC₅₀ value of 8.6 μM±0.10 log units. The reduced potency of 1 in inhibiting 5-LO product formation in neutrophils could be due to hampered cellular uptake of the compound or competition with endogenous factors. Still, when supplementing neutrophils with 1 mM dithiothreitol, 1 inhibited 5-LO with an IC₅₀ value of 0.42 μM±0.10 log units, which is in accordance with data from the cell-free assay. Thus, 1 is likely not fully reduced in the native neutrophil environment. Moreover, 1 potently inhibited 5-LO in neutrophil homogenates with 1 mM dithiothreitol with an IC₅₀ value of 85.5 nM±0.13 log units (FIG. 2c), which is in line with the cell-free 5-LO assay. Hence, our data not only shows that hydroquinone 1 is the active species against 5-LO, but also reinforces there is insufficient hydroquinone formation within neutrophils. However, from an anticancer activity standpoint, the marked reducing environment within cancer cells and microenvironment, e.g. through glutathione or NQO1, may present the ideal setting for phenotype modulation by 1 in cells with 5-LO overexpression.

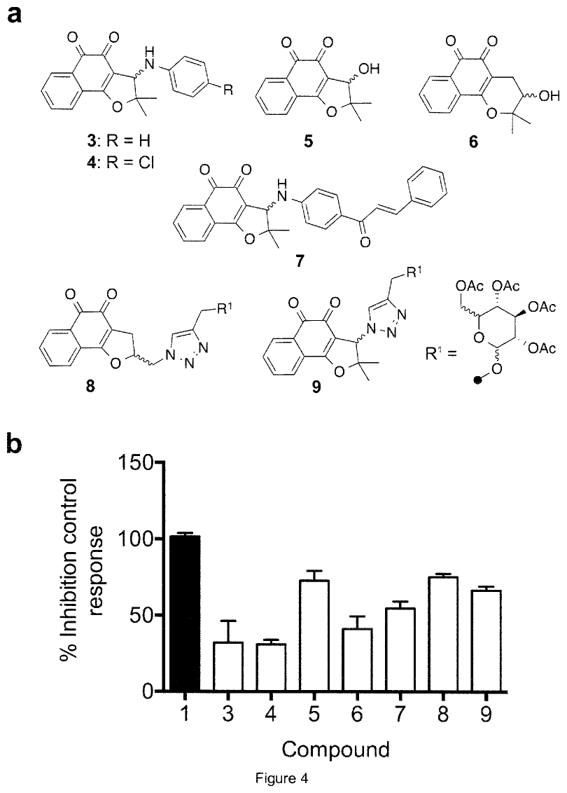

To probe for metaloenzyme selectivity, we screened 1 against 12-LO and 15-LO-2 using two different methods. Weak inhibition (IC50>30 μM) was obtained in both cases, which highlights selectivity of 1 for 5-LO (FIG. 3). Screening against the solvent-exposed Zn2+-containing phosphodiesterase 5 (PDE5) also revealed inactivity, suggesting that 1 is not a general metal chelator. Overall, we provide robust evidence that 1 is a true 5-LO inhibitor. Notably, the scaffold of 1 is not exploited among 5-LO ligands despite preserving the pharmacophore features for target engagement. Thus, similarity searches with commonly used fingerprints would likely fail in identifying the 1-5-LO relationship despite the existence of other ortho-quinone 5-LO inhibitors, i.e. the ligand-target association is not apparent. To further probe the specific 5-LO recognition, we expanded our hit by synthesizing a focused library of racemic β-lapachone-inspired entities. A range of inhibition potencies were obtained in cell-free 5-LO assays, supporting the importance of the substitution pattern for bioactivity and the specific, directed interactions of 1 with 5-LO (FIG. 4).

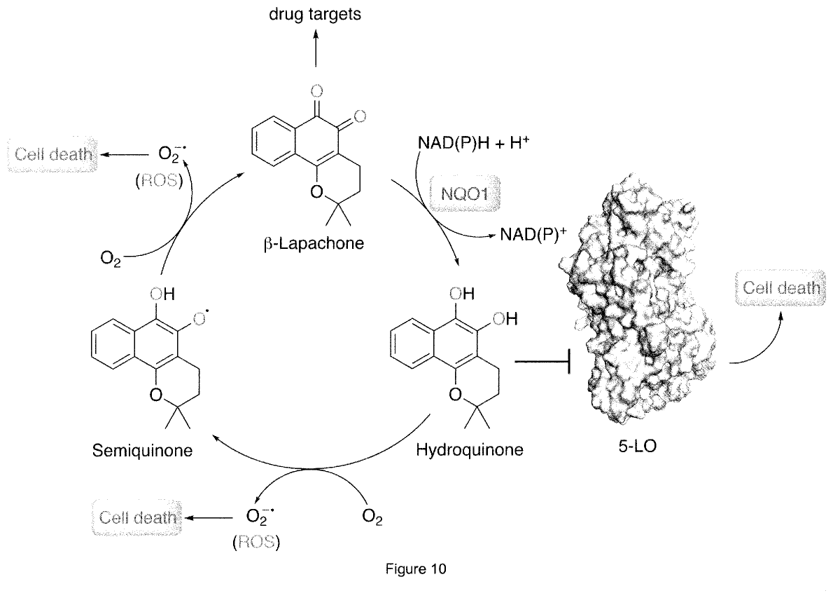

Next, we confirmed that activity of 5-LO could be reinstated upon wash-out of 1, and that a variation of arachidonic acid concentration (2.5-40 μM) led only to minor alterations of the potency of 1. Taken together, our data robustly advocates for a reversible and allosteric modulation of 5-LO by hydroquinone 1, as a potential means for mediating the anticancer activity.³⁹ This result not only agrees with the absence of modulation of related (12-/15-LO) and unrelated (PDE5) metal-containing targets, but also contrasts with a report suggesting chelation of the Fe³⁺ centre in IDO1.⁵³ Our results also show that β-lapachone requires to be reduced to its hydroquinone form (e.g. through NQO1 in cancer cells) in order to modulate 5-LO (FIG. 10).

In agreement with our previous data, 1 expands the 5-LO inhibitor space, as it is structurally unrelated to known chemical matter (Nearest neighbor: CHEMBL275120³⁹ Tanimoto index=0.23; All 5-LO ligands: Average Tanimoto index=0.11±0.03; Morgan Fingerprints, radius 2, 2048 bits), yet preserves the required pharmacophore features for target engagement.

To probe the specific 5-LO recognition we expanded our hit by synthesizing a focused library of racemic β-lapachone-inspired naphthoquinones (FIG. 4a). The compounds were prepared as previously reported.²¹⁻²³ Generally, the in situ bromination of an appropriate starting material afforded the key intermediates, which were subsequently functionalized with the required nucleophilic species. A range of functional inhibition potencies were obtained (FIG. 4b), supporting the specific, directed interactions by these small molecules with 5-LO. For example, inhibition of 5-LO appears to be sensitive to ring contraction/expansion, and is not solely dependent on presence of the redox center. We further screened 1 at a concentration of 5 μM against the homologous 12-LO and 15-LO-2 counterparts, obtaining weak to no inhibition, i.e. 38% and −13%, respectively. Moreover, screening of 1 against the Zn²⁺-containing phosphodiesterase 5 revealed inactivity at 2 μM, suggesting that 1 is not a general metal chelator in protein systems with solvent-accessible metal-dependent catalytic centres.²⁴

As counter screen, we tested 1 against gram negative (Escherichia coli) and gram-positive (Staphylococcus aureus) bacteria, given their susceptibility to oxidative stress induced by reactive oxygen species-producing chemical entities.²⁵⁻²⁸ A minimum inhibitory concentration of 60 μM against S. aureus and a minimum bactericidal concentration above 200 μM against both bacteria were obtained. Our data thus supports the absence of general target modulation by 1.

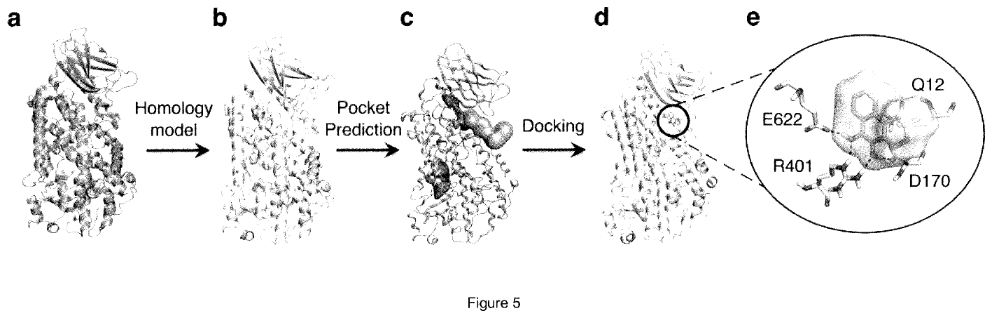

To obtain insights into the putative binding mode of 1 to 5-LO we built homology models for the wild type 5-LO (FIG. 5a,b). Based on the observation of high 5-LO flexibility upon binding of arachidonic acid, we used both liganded and apo mutant 5-LO structures as templates. We then predicted binding pockets with volume>110 Å³ for all 5-LO models using MOE 2016.10 (Chemical Computing Group, Canada), given that only a small fraction of pockets with smaller volumes accommodate ligands (FIG. 5c).²⁹

Next, we generated 50 decoys with DUD-E³⁰ using 1 as seed structure. These were further processed with SPiDER on the assumption that compounds structurally unrelated to 1, and pharmacophorically distinct from known 5-LO inhibitors present low binding likelihood.

A total of 21 small molecules fulfilled both criteria, advocating for the structural diversity among 5-LO inhibitors and potential limitations of using purely chemical structure-based descriptors for decoy selection. The decoys and 1 were subsequently docked with GOLD³¹ into all predicted pockets and enrichment factors were calculated for pocket prioritization, together with visual inspection of the docked poses (FIG. 5d-e). The resulting model suggests that hydroquinone 1 binds through hydrogen bonds to D170, R401 and E622, and performs an amide-π stacking with Q12, at the C2-like and catalytic domains' interface. Disruption of the C2-catalytic domain interaction is known to increase 5-LO activity,⁵⁴ which can be counteracted by hydroquinone

Our molecular docking simulations suggest that 1 has low probability of interacting with the iron center or dock in its vicinity, which is fully supported by our enzymatic data. Accordingly, 1 is predicted to sit in a transient allosteric site between the C2-like and catalytic domains through interaction with W13. Hanke et al.²⁹ had previously postulated binding of pirinixic acid derivatives in the same loop—a region well known to accommodate phospholipids and being critical for 5-LO function and dynamics.^{32,33} Alignment of 5-LO, 12-LO and 15-LO-2 sequences shows substantial dissimilarities in the predicted binding pocket region, namely the absence of W13 for the latter two, which may explain the weak effects of 1 against those. A molecular dynamics simulation of the 1-5-LO complex showed moderate stability of the interaction, in line with the observed inhibitory activity. The result also pinpoints that that a transient binding site not sampled in the structural model cannot be excluded.

β-Lapachone may itself modulate hitherto unknown drug targets while the generated reactive oxygen species in the redox cycle are also accountable for the phenotypic effects. Indeed, β-lapachone binds to 5-LO competitively to phosphatidylcholine, which is known to increase the catalytic activity of 5-LO. The predicted binding pose suggests no interaction with tryptophan residues. Because tryptophan residues display fluorescence under certain experimental conditions, we challenged our binding model by monitoring tryptophan fluorescence; wherein a blue shift indicates a binding interaction. Using purified human 5-LO and supplementing it with 1 mM dithiothreitol to ensure reduction of β-lapachone to the corresponding hydroquinone we observed a blue shift in tryptophan fluorescence with increasing concentrations of 1, suggesting progressive burial of a tryptophan residue upon binding of the NP. Of note, 1 does not present auto-fluorescence at the monitored wavelength, ruling out artifactual readouts. Further inspection of the binding model shows that the remaining 14 tryptophan residues are located in shallow, non-ligandable surfaces, supporting W13 as a potential binding counterpart for 1.

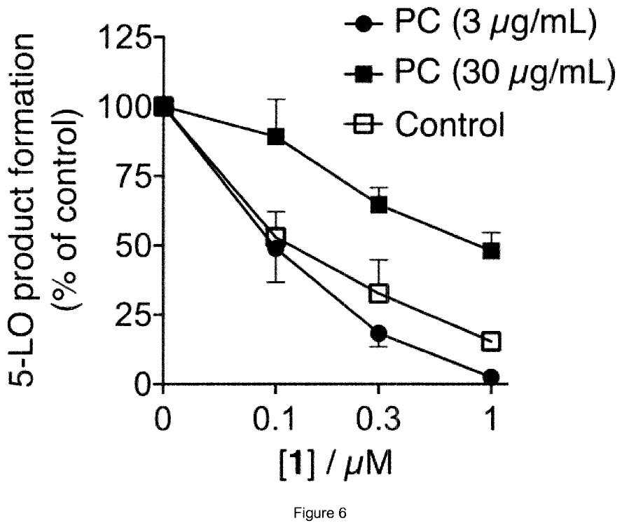

We next performed competition assays between 1 and phosphatidylcholine, which binds to the predicted groove. Increasing concentrations of phosphatidylcholine were found to promote the 5-LO product formation and markedly diminish the 5-LO blocking efficiency of hydroquinone 1 (FIG. 6), providing evidence for competitive binding. Furthermore, an alignment of 5-LO, 12-LO and 15-LO-2 sequences showed dissimilarities in the predicted binding pocket region, which may explain the weak effects of 1 against the latter two. Altogether, the excellent agreement between the in silico models and biochemical data suggests a direct interaction between hydroquinone 1 and 5-LO, through a hitherto unknown allosteric recognition mechanism at the C2-catalytic domains' interface.

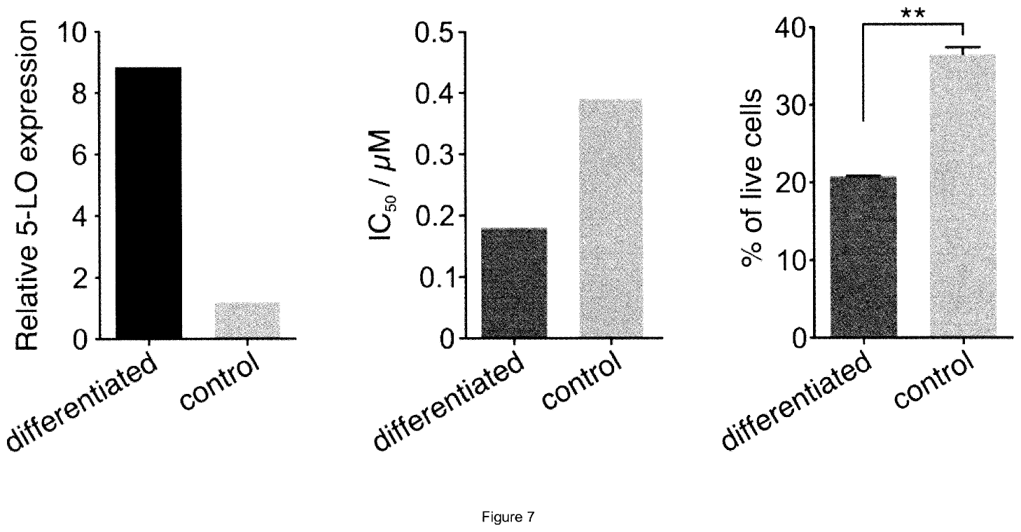

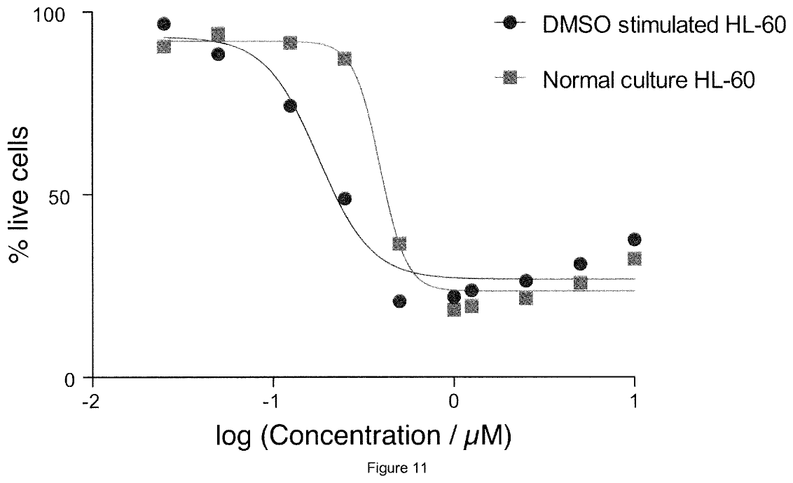

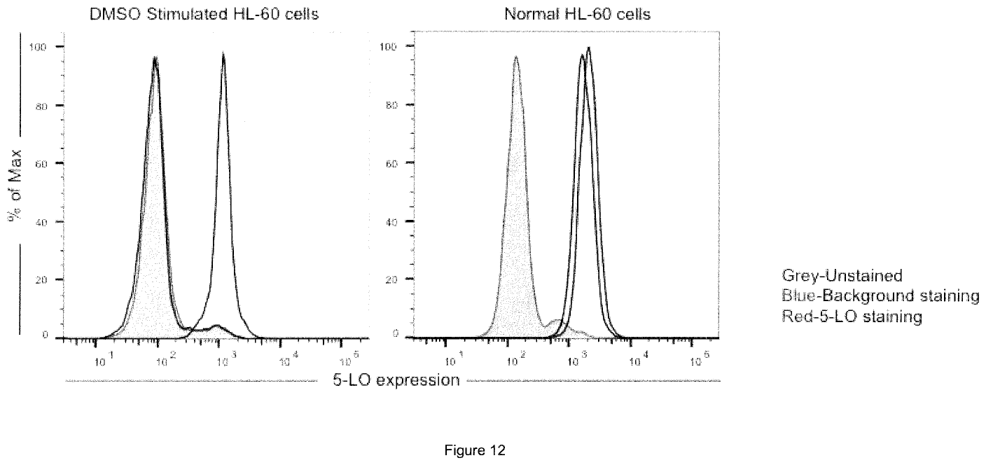

To ascertain the importance of allosteric binding and 5-LO inhibition for the anticancer activity of 1 we conducted cell-viability assays with the HL-60 cell line. This leukemia cell line does not overexpress 5-LO except when differentiated (FIGS. 7 and 12).^{56, 57} Treatment of both groups with 1 showed that cells overexpressing 5-LO were more sensitive than the control (FIG. 7 centre panel, FIG. 11), displaying IC₅₀ values of 0.18 μM and 0.39 μM, respectively. Indeed, the result is statistically significant (FIG. 7 right panel), which endorses 5-LO as an anticancer target for 1 in vitro (FIG. 10). The result ultimately confirms our hypothesis that in cancer cells the strongly reducing environment efficiently generates hydroquinone 1 for 5-LO modulation.

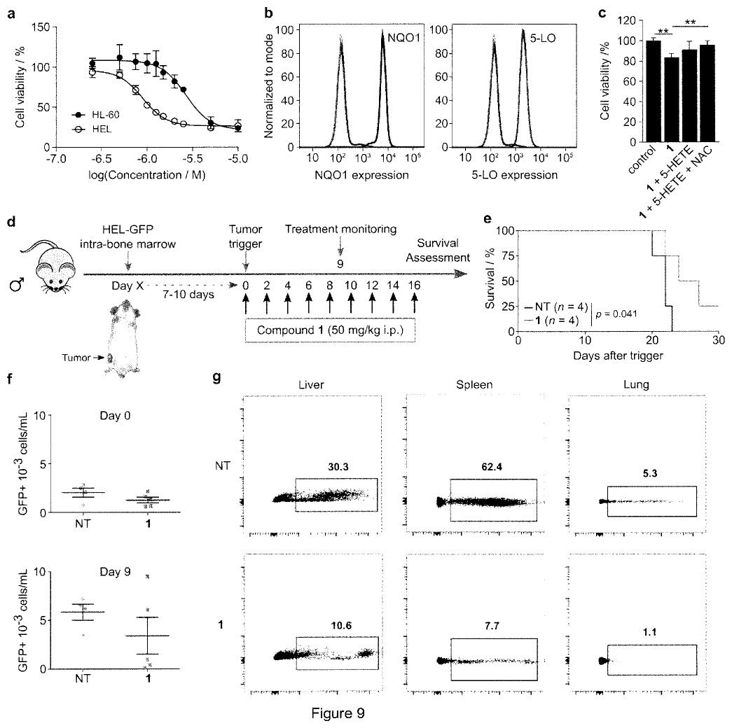

Compound 1 had been previously assessed for anticancer activity against the NCI-60 panel of human cancer cell lines.³⁴ For analysis, we retrieved the z-transformed pGI₅₀ values, whereby high z values correspond to high cell line sensitivity to 1. Compound 1 strongly modulated the viability of blood and skin cancers (FIG. 9a), including the AML cell line HL-60 (pGI₅₀-score=2.38, GI₅₀=62.4 nM).

Since the NCI-60 panel is profiled for mRNA expression³⁵ and drug sensitivity, we correlated both data to leverage our understanding of the mechanisms of anticancer activity for 1. Our data fully corroborates the engagement of multiple targets as previously predicted. By ranking genes according to their expression and Glo correlation, and subsequently applying gene set enrichment analysis,³⁶ we were able to identify pathways mediating both anticancer activity and drug resistance.

Motivated by the engagement of 5-LO by 1 we explored how this ligand-target pair can be harnessed in a cancer context. We identified that both 5-LO and NQO1 are expressed in blood cancer cell lines and in AML. Hence, our data suggests a complementary and hitherto unexploited mechanism of action for the anticancer activity of 1.

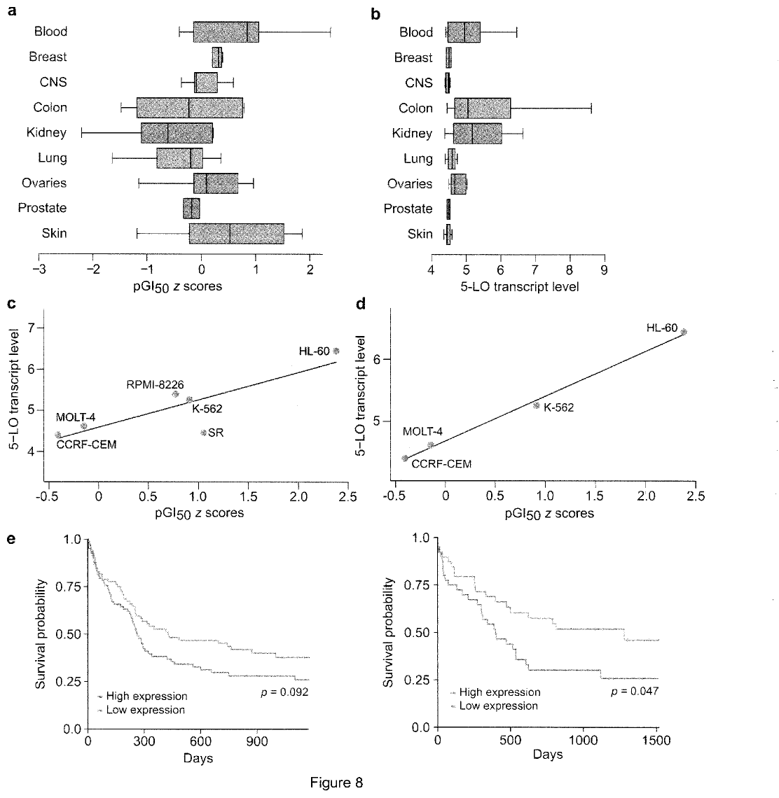

We investigated the correlation between cancer activity and 5-LO expression. Indeed, a strong positive correlation (Pearson correlation coefficient=0.85, p=0.03, FIG. 8c) was found, with the leukemia cell line HL-60 being the most sensitive, and where 5-LO is most expressed. Interestingly, re-analysis only on leukemia cell lines (CCRF-CEM, HL-60, K-562 and MOLT-4), afforded a higher correlation (Pearson correlation coefficient=1, p=0.002, FIG. 8d). Most importantly, a survival analysis using a log-rank test revealed the prognostic value of 5-LO in two independent AML cohorts³⁷ i.e., high 5-LO expression is associated with lower patient survival (p=0.092 and 0.047, FIG. 8e). Furthermore, we found a similar correlation for chronic lymphocytic leukemia samples³⁸ (p=0.002). Altogether our data unveils the clinical importance of 5-LO modulation in a leukemia context, and ascertains it as a relevant macromolecular target for the bioactivity of 1.

We then set out to experimentally leverage our in silico findings and models. In our hands, compound 1 displayed and IC₅₀ value of 2.7 μM±0.04 log units against HL-60 (95% confidence interval=2.2-3.2 μM; FIG. 9a), which we confirmed to express both 5-LO and NQO1 (FIG. 9b). Moreover, rescue of 5-LO inhibition by 1 in HL-60 cells with 5-HETE lactone, a 5-LO metabolite, showed a tendency for improved survival, which was significant upon addition of 1 μM of the reactive oxygen species (ROS) quencher N-acetylcysteine (NAC, FIG. 9c). While NAC counteracts ROS originating from bioactivation of 1 by NQO1, it has now been established that selective 5-LO inhibitors also lead to formation of ROS, which mediate early apoptotic events.³⁹ Our data thus suggests that both mechanisms of action are relevant in an AML context.

To evaluate the anti-AML efficacy of 1 in vivo, we injected HEL cells (IC₅₀ (1)=0.9 μM±0.02 log units; 95% confidence interval=0.8-1.0 μM, FIG. 9a) that are refractory to the standard-of-care AML therapy into the right tibia of irradiated NSG mice, and initiated the treatment upon tumor trigger in the blood (FIG. 9d). We observed that intra-peritoneal administration of 1 at 50 mg/kg significantly increased overall survival in this model (FIG. 9e). Assessment of tumour burden in the blood at day 9, in the middle of the treatment rounds, showed a tendency for lower burden upon treatment with 1, although not statistically significant (FIG. 9f). In fact, the mouse treated with 1 that lived the longest, and which was sacrificed at day 30 in the absence of detectable symptoms, presented lower tumour burden in all the analysed organs (liver, spleen and lung) when compared to any of the non-treated control mice (FIG. 9g). Our subsequent efforts to test the efficacy of treatment with 1 at higher doses (starting at 100 mg/kg) were compromised by its toxicity upon intra-peritoneal or intravenous injection. Nevertheless, these results constitute an important proof-of-concept for future development of tumour-specific, and thus safer, delivery strategies for 1 in vivo.

NPs with anticancer activity have traditionally served as excellent starting points for development of life-changing therapies. Though, harnessing their potential as drug leads has been a challenging and daunting task provided that the underlying mechanisms of action and polypharmacology networks are largely unknown. To that end, ligand-based machine learning tools offer an expeditious and cost-effective solution to prioritize screening campaigns. The naphthoquinone 1 has attracted considerable attention as a drug candidate, being actively pursued in clinical trials. Using an in silico, data mining platform, we disclosed an unprecedented link between allosteric modulation of 5-LO and the anticancer activity of 1. Crucially, 1 displayed significant effects in an in vivo model of disseminated disease that is refractory to current standard-of-care chemotherapy. Importantly, 5-LO may be used as marker for prognosis of AML and CLL.

### REFERENCES

(1) Harvey, A. L.; Edrada-Ebel, R.; Quinn, R. J. The re-emergence of natural products for drug discovery in the genomics era. Nat. Rev. Drug Discov. 2015, 14, 111-129.
(2) Rodrigues, T.; Reker, D.; Kunze, J.; Schneider, P.; Schneider, G. Revealing the Macromolecular Targets of Fragment-Like Natural Products. Angew. Chem. Int. Ed. 2015, 54, 10516-10520.
(3) Rodrigues, T.; Reker, D.; Schneider, P.; Schneider, G. Counting on natural products for drug design. Nat. Chem. 2016, 8, 531-541.
(4) Reker, D.; Perna, A. M.; Rodrigues, T.; Schneider, P.; Reutlinger, M.; Monch, B.; Koeberle, A.; Lamers, C.; Gabler, M.; Steinmetz, H.; Muller, R.; Schubert-Zsilavecz, M.; Werz, O.; Schneider, G. Revealing the macromolecular targets of complex natural products. Nat. Chem. 2014, 6, 1072-1078.
(5) Rollinger, J. M.; Schuster, D.; Danzl, B.; Schwaiger, S.; Markt, P.; Schmidtke, M.; Gertsch, J.; Raduner, S.; Wolber, G.; Langer, T.; Stuppner, H. In silico target fishing for rationalized ligand discovery exemplified on constituents of Ruta graveolens. Planta Med. 2009, 75, 195-204.
(6) Roche, O.; Schneider, P.; Zuegge, J.; Guba, W.; Kansy, M.; Alanine, A.; Bleicher, K.; Danel, F.; Gutknecht, E. M.; Rogers-Evans, M.; Neidhart, W.; Stalder, H.; Dillon, M.; Sjogren, E.; Fotouhi, N.; Gillespie, P.; Goodnow, R.; Harris, W.; Jones, P.; Taniguchi, M.; Tsujii, S.; von der Saal, W.; Zimmermann, G.; Schneider, G. Development of a virtual screening method for identification of “frequent hitters” in compound libraries. J. Med. Chem. 2002, 45, 137-142.
(7) Baell, J. B.; Holloway, G. A. New substructure filters for removal of pan assay interference compounds (PAINS) from screening libraries and for their exclusion in bioassays. J. Med. Chem. 2010, 53, 2719-2740.
(8) Pouliot, M.; Jeanmart, S. Pan Assay Interference Compounds (PAINS) and Other Promiscuous Compounds in Antifungal Research. J. Med. Chem. 2016, 59, 497-503.
(9) Baell, J. B. Feeling Nature's PAINS: Natural Products, Natural Product Drugs, and Pan Assay Interference Compounds (PAINS). J. Nat. Prod. 2016, 79, 616-628.
(10) Clemons, P. A.; Bodycombe, N. E.; Carrinski, H. A.; Wilson, J. A.; Shamji, A. F.; Wagner, B. K.; Koehler, A. N.; Schreiber, S. L. Small molecules of different origins have distinct distributions of structural complexity that correlate with protein-binding profiles. Proc. Natl. Acad. Sci. U.S.A. 2,010, 107, 18787-18792.
(11) Reinicke, K. E.; Bey, E. A.; Bentle, M. S.; Pink, J. J.; Ingalls, S. T.; Hoppel, C. L.; Misico, R. I.; Arzac, G. M.; Burton, G.; Bornmann, W. G.; Sutton, D.; Gao, J.; Boothman, D. A. Development of beta-lapachone prodrugs for therapy against human cancer cells with elevated NAD(P)H:quinone oxidoreductase 1 levels. Clin. Cancer Res. 2005, 11, 3055-3064.
(12) Lipinski, C. A. Rule of five in 2015 and beyond: Target and ligand structural limitations, ligand chemistry structure and drug discovery project decisions. Adv. Drug Deliv. Rev. 2016, 101, 34-41.
(13) Walters, W. P.; Murcko, A. A.; Murcko, M. A. Recognizing molecules with drug-like properties. Curr. Opin. Chem. Biol. 1999, 3, 384-387.
(14) Reker, D.; Rodrigues, T.; Schneider, P.; Schneider, G. Identifying the macromolecular targets of de novo-designed chemical entities through self-organizing map consensus. Proc. Natl. Acad. Sci. U.S.A. 2014, 111, 4067-4072.
(15) Rodrigues, T.; Sieglitz, F.; Somovilla, V. J.; Cal, P. M.; Galione, A.; Corzana, F.; Bernardes, G. J. Unveiling (−)-Englerin A as a Modulator of L-Type Calcium Channels. Angew. Chem. Int. Ed. 2016, 55, 11077-11081.
(16) Nickel, J.; Gohlke, B. O.; Erehman, J.; Banerjee, P.; Rong, W. W.; Goede, A.; Dunkel, M.; Preissner, R. SuperPred: update on drug classification and target prediction. Nucleic Acids Res. 2014, 42, W26-31.
(17) Keiser, M. J.; Roth, B. L.; Armbruster, B. N.; Ernsberger, P.; Irwin, J. J.; Shoichet, B. K. Relating protein pharmacology by ligand chemistry. Nat. Biotechnol. 2007, 25, 197-206.
(18) Li, C. J.; Averboukh, L.; Pardee, A. B. beta-Lapachone, a novel DNA topoisomerase I inhibitor with a mode of action different from camptothecin. J. Biol. Chem. 1993, 268, 22463-22468.
(19) Lee, J. H.; Cheong, J.; Park, Y. M.; Choi, Y. H. Down-regulation of cyclooxygenase-2 and telomerase activity by beta-lapachone in human prostate carcinoma cells. Pharmacol. Res. 2005, 51, 553-560.
(20) Janusz, J. M.; Young, P. A.; Ridgeway, J. M.; Scherz, M. W.; Enzweiler, K.; Wu, L. I.; Gan, L.; Darolia, R.; Matthews, R. S.; Hennes, D.; Kellstein, D. E.; Green, S. A.; Tulich, J. L.; Rosario-Jansen, T.; Magrisso, I. J.; Wehmeyer, K. R.; Kuhlenbeck, D. L.; Eichhold, T. H.; Dobson, R. L.; Sirko, S. P.; Farmer, R. W. New cyclooxygenase-2/5-lipoxygenase inhibitors. 1. 7-tert-butyl-2,3-dihydro-3,3-dimethylbenzofuran derivatives as gastrointestinal safe antiinflammatory and analgesic agents: discovery and variation of the 5-keto substituent. J. Med. Chem. 1998, 41, 1112-1123.
(21) da Silva Junior, E. N.; de Souza, M. C.; Pinto, A. V.; Pinto Mdo, C.; Goulart, M. O.; Barros, F. W.; Pessoa, C.; Costa-Lotufo, L. V.; Montenegro, R. C.; de Moraes, M. O.; Ferreira, V. F. Synthesis and potent antitumor activity of new arylamino derivatives of nor-beta-lapachone and nor-alpha-lapachone. Bioorg. Med. Chem. 2007, 15, 7035-7041.
(22) da Silva Junior, E. N.; de Souza, M. C.; Fernandes, M. C.; Menna-Barreto, R. F.; Pinto Mdo, C.; de Assis Lopes, F.; de Simone, C. A.; Andrade, C. K.; Pinto, A. V.; Ferreira, V. F.; de Castro, S. L. Synthesis and anti-Trypanosoma cruzi activity of derivatives from nor-lapachones and lapachones. Bioorg. Med. Chem. 2008, 16, 5030-5038.
(23) Jardim, G. A. M.; Guimaraes, T. T.; Pinto, M. d. C. F. R.; Cavalcanti, B. C.; Farias, K. M. d.; Pessoa, C.; Gatto, C. C.; Nair, D. K.; Namboothiri, I. N. N.; Junior, E. N. d. S. Naphthoquinone-based chalcone hybrids and derivatives: synthesis and potent activity against cancer cell lines. Med. Chem. Commun. 2015, 6, 120-130.
(24) Owen, D. R.; Walker, J. K.; Jon Jacobsen, E.; Freskos, J. N.; Hughes, R. O.; Brown, D. L.; Bell, A. S.; Brown, D. G.; Phillips, C.; Mischke, B. V.; Molyneaux, J. M.; Fobian, Y. M.; Heasley, S. E.; Moon, J. B.; Stallings, W. C.; Joseph Rogier, D.; Fox, D. N.; Palmer, M. J.; Ringer, T.; Rodriquez-Lens, M.; Cubbage, J. W.; Blevis-Bal, R. M.; Benson, A. G.; Acker, B. A.; Maddux, T. M.; Tollefson, M. B.; Bond, B. R.; Macinnes, A.; Yu, Y. Identification, synthesis and SAR of amino substituted pyrido[3,2b]pyrazinones as potent and selective PDE5 inhibitors. Bioorg. Med. Chem. Lett. 2009, 19, 4088-4091.
(25) Imlay, J. A. The molecular mechanisms and physiological consequences of oxidative stress: lessons from a model bacterium. Nat. Rev. Microbiol. 2013, 11, 443-454.
(26) Medina, L. F.; Hertz, P. F.; Stefani, V.; Henriques, J. A.; Zanotto-Filho, A.; Brandelli, A. Aminonaphthoquinone induces oxidative stress in Staphylococcus aureus. Biochem. Cell Biol. 2006, 84, 720-727.
(27) Becerra, M. C.; Albesa, I. Oxidative stress induced by ciprofloxacin in Staphylococcus aureus. Biochem. Biophys. Res. Commun. 2002, 297, 1003-1007.
(28) Goswami, M.; Mangoli, S. H.; Jawali, N. Involvement of reactive oxygen species in the action of ciprofloxacin against Escherichia coli. Antimicrob. Agents Chemother. 2006, 50, 949-954.
(29) Hanke, T.; Dehm, F.; Liening, S.; Popella, S. D.; Maczewsky, J.; Pillong, M.; Kunze, J.; Weinigel, C.; Barz, D.; Kaiser, A.; Wurglics, M.; Lammerhofer, M.; Schneider, G.; Sautebin, L.; Schubert-Zsilavecz, M.; Werz, O. Aminothiazole-featured pirinixic acid derivatives as dual 5-lipoxygenase and microsomal prostaglandin E2 synthase-1 inhibitors with improved potency and efficiency in vivo. J. Med. Chem. 2013, 56, 9031-9044.
(30) Mysinger, M. M.; Carchia, M.; Irwin, J. J.; Shoichet, B. K. Directory of useful decoys, enhanced (DUD-E): better ligands and decoys for better benchmarking. J. Med. Chem. 2012, 55, 6582-6594.
(31) Jones, G.; Willett, P.; Glen, R. C. Molecular recognition of receptor sites using a genetic algorithm with a description of desolvation. J. Mol. Biol. 1995, 245, 43-53.
(32) Hornig, C.; Albert, D.; Fischer, L.; Hornig, M.; Radmark, O.; Steinhilber, D.; Werz, O. 1-Oleoyl-2-acetylglycerol stimulates 5-lipoxygenase activity via a putative (phospho)lipid binding site within the N-terminal C2-like domain. J. Biol. Chem. 2005, 280, 26913-26921.
(33) Kulkarni, S.; Das, S.; Funk, C. D.; Murray, D.; Cho, W. Molecular basis of the specific subcellular localization of the C2-like domain of 5-lipoxygenase. J. Biol. Chem. 2002, 277, 13167-13174.
(34) Shoemaker, R. H. The NCI60 human tumour cell line anticancer drug screen. Nat. Rev. Cancer 2006, 6, 813-823.
(35) Gmeiner, W. H.; Reinhold, W. C.; Pommier, Y. Genome-wide mRNA and microRNA profiling of the NCI 60 cell-line screen and comparison of FdUMP[10] with fluorouracil, floxuridine, and topoisomerase 1 poisons. Mol. Cancer Ther. 2010, 9, 3105-3114.
(36) Subramanian, A.; Tamayo, P.; Mootha, V. K.; Mukherjee, S.; Ebert, B. L.; Gillette, M. A.; Paulovich, A.; Pomeroy, S. L.; Golub, T. R.; Lander, E. S.; Mesirov, J. P. Gene set enrichment analysis: a knowledge-based approach for interpreting genome-wide expression profiles. Proc. Natl. Acad. Sci. U.S.A. 2005, 102, 15545-15550.
(37) Metzeler, K. H.; Hummel, M.; Bloomfield, C. D.; Spiekermann, K.; Braess, J.; Sauerland, M. C.; Heinecke, A.; Radmacher, M.; Marcucci, G.; Whitman, S. P.; Maharry, K.; Paschka, P.; Larson, R. A.; Berdel, W. E.; Buchner, T.; Wormann, B.; Mansmann, U.; Hiddemann, W.; Bohlander, S. K.; Buske, C.; Cancer; Leukemia Group, B.; German, A. M. L. C. G. An 86-probe-set gene-expression signature predicts survival in cytogenetically normal acute myeloid leukemia. Blood 2008, 112, 4193-4201.
(38) Herold, T.; Jurinovic, V.; Metzeler, K. H.; Boulesteix, A. L.; Bergmann, M.; Seiler, T.; Mulaw, M.; Thoene, S.; Dufour, A.; Pasalic, Z.; Schmidberger, M.; Schmidt, M.; Schneider, S.; Kakadia, P. M.; Feuring-Buske, M.; Braess, J.; Spiekermann, K.; Mansmann, U.; Hiddemann, W.; Buske, C.; Bohlander, S. K. An eight-gene expression signature for the prediction of survival and time to treatment in chronic lymphocytic leukemia. Leukemia 2011, 25, 1639-1645.
(39) Ghosh, J.; Myers, C. E. Inhibition of arachidonate 5-lipoxygenase triggers massive apoptosis in human prostate cancer cells. Proc. Natl. Acad. Sci. U.S.A. 1998, 95, 13182-13187.
(40) Gilbert, N. C.; Rui, Z.; Neau, D. B.; Waight, M. T.; Bartlett, S. G.; Boeglin, W. E.; Brash, A. R.; Newcomer, M. E. Conversion of human 5-lipoxygenase to a 15-lipoxygenase by a point mutation to mimic phosphorylation at Serine-663. FASEB J. 2012, 26, 3222-3229.
(41) Goswami, C. P.; Nakshatri, H. PROGgeneV2: enhancements on the existing database. BMC cancer 2014, 14, 970.
(42) Hooker, S. C. The constitution of lapachol and its derivatives. Part V. The structure of paternò's “isolapachone”. J. Am. Chem. Soc. 58, 1190-1197 (1936).
(43) Schaffner-Sabba, K., Schmidt-Ruppin, K. H., Wehrli, W., Schuerch, A. R. & Wasley, J. W. Beta-Lapachone: synthesis of derivatives and activities in tumor models. J. Med. Chem. 27, 990-994 (1984).
(44) Silva, R. S. et al. Synthesis of naphthofuranquinones with activity against Trypanosom26a cruzi. Eur. J. Med. Chem. 41, 526-530 (2006).
(45) da Silva Junior, E. N. et al. Synthesis and anti-Trypanosoma cruzi activity of derivatives from nor-lapachones and lapachones. Bioorg. Med. Chem. 16, 5030-5038 (2008).
(46) Sun, J. S., Geiser, A. H. & Frydman, B. A preparative synthesis of lapachol and related naphthoquinones. Tetrahedron Lett. 39, 8221-8224 (1998).
(47) Salas, C. et al. Trypanosoma cruzi: activities of lapachol and alpha- and beta-lapachone derivatives against epimastigote and trypomastigote forms. Bioorg. Med. Chem. 16, 668-674 (2008).
(48) Rocha, D. R. d. et al. Synthesis of fused chromene-1,4-naphthoquinones via ring-closing metathesis and Knoevenagel-electrocyclization under acid catalysis and microwave irradiation. Tetrahedron 70, 3266-3270 (2014)
(49) Niehues, M. et al. Biomimetic in vitro oxidation of lapachol: a model to predict and analyse the in vivo phase I metabolism of bioactive compounds. Eur. J. Med. Chem. 54, 804-812 (2012).
(50) Sacau, E. P. et al. Inhibitory effects of lapachol derivatives on Epstein-Barr virus activation. Bioorg. Med. Chem. 11, 483-488 (2003).
(51) M. Reutlinger et al. Mol. Inf., 2013, 32, 133
(52) D. Reker et al, Proc. Natl Acad. Sci. U.S.A., 2014, 111, 4067.
(53) H. E. Flick et al Int. J. Tryptophan Res., 2013, 6, 35.
(54) M. Rakonjac Ryge et al Arch. Biochem. Biophys., 2014, 545, 179
(55) G. Piechele et al Pharmacol. Res., 1993, 27, 53.
(56) M. Brungs et al Biochem. Biophys. Res. Commun., 1994, 205, 1572

1. A method of treating cancer characterized by 5-lipoxygenase (5-LO) expression comprising;
administering a β-lapachone compound to a patient in need thereof.

2. A method according to claim 1 wherein the β-lapachone compound is a reversible, allosteric antagonist of 5-LO.

3. A method according to claim 1 or claim 2 wherein the β-lapachone compound is β-lapachone or an analogue, derivative or prodrug thereof.

4. A method according to claim 3 wherein the β-lapachone compound has the formula (A): 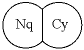
wherein:
Nq is optionally substituted naphthoquinone;
Cy is an optionally substituted heterocyclic ring fused to Nq;
and salts, solvates and protected forms thereof.

5. The method according to claim 4, wherein Cy is fused to the quinone ring of the naphthoquinone.

6. The method according to claim 4 or claim 5, wherein Nq is naphthoquinone.

7. The method according to any one of claims 4 to 6, wherein Nq is 1,2-naphthoquinone or 1,4-naphthoquinone, such as 1,2-naphthoquinone.

8. The method according to any one of claims 4 to 7, wherein Cy, including the two carbon atoms from the naphthoquinone group to which it is fused, has 5, 6 or 7 ring atoms, such as 5 or 6 ring atoms, such as 5 ring atoms.

9. The method according to any one of claims 4 to 8, wherein Cy is an oxygen-containing heterocycle.

10. The method according to claim 9, wherein Cy, including the two carbon atoms from the naphthoquinone group to which it is fused, is a 2,3-dihydrofuran group or a 2,5-dihydrofuran group, such as a 2,3-dihydrofuran group.

11. The method according to claim 9, wherein Cy, including the two carbon atoms from the naphthoquinone group to which it is fused, is a 3,4-dihydro-2H-pyran group or a 3,6-dihydro-2H-pyran group, such as a 3,4-dihydro-2H-pyran group.

12. The method according to any one of claims 4 to 11, wherein Cy is optionally substituted with one or more substituent groups, —R^{Cy}, selected from the group consisting of —OH, —SH, —NH₂, halo, and -L¹-R⁴, where -L¹- is selected from a covalent bond, alkylene, —O—, —S—, —N(H)—, and —N(R^{L1})—, where —R^{L1} is alkyl, and —R⁴ is selected from the group consisting of alkyl, cycloalkyl, heterocyclyl, and aryl, where each of the alkyl, cycloalkyl, heterocyclyl, and aryl is independently optionally substituted.

13. The method according to claim 12, wherein Cy is optionally substituted with —OH and -L¹-R⁴, and is optionally further substituted with one or more substituent groups —R^{Cy}.

14. The method according to claim 12, wherein Cy is substituted with two alkyl substituent groups, such as two methyl groups, and is optionally further substituted with one or more substituent groups —R^{Cy}, such as one more substituent —R^{Cy}, such as —OH or -L¹-R⁴.

15. A method according to any one of claims 1 to 4 wherein the β-lapachone compound is β-lapachone.

16. A method according to any one of claims 1 to 4 wherein the β-lapachone compound is an imine protected form, such as an optionally-substituted phenylimine protected form, such as the Q-lapachone compound is ARQ 761.

17. The method according to claim 3 wherein the β-lapachone compound has the formula (D): 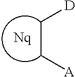
wherein:
Nq is optionally substituted naphthoquinone;
-A is selected from —OH, —SH, —NH₂ and —NHR, where R is selected from alkyl, aryl and aralkyl;
-D is an optionally substituted alkenyl group,
and salts, solvates and protected forms thereof.

18. The method according to claim 17, wherein -A is —OH.

19. The method according to claim 17 or claim 18, wherein Nq is naphthoquinone.

20. The method according to any one of claims 17 to 19, wherein Nq is 1,2-naphthoquinone or 1,4-naphthoquinone, such as 1,2-naphthoquinone.

21. The method according to any one of claims 17 to 20, wherein -D is: 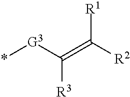
where -G³- is a covalent bond or alkylene, and each of —R¹, —R², —R³ is independently —R^{Cy}, where each —R^{Cy} is selected from the group consisting of —OH, —SH, —NH₂, halo, and -L¹-R⁴, where -L¹-is selected from a covalent bond, alkylene, —O—, —S—, —N(H)—, and —N(R^{L1})—, where —R^{L1} is alkyl, and —R⁴ is selected from the group consisting of alkyl, cycloalkyl, heterocyclyl, and aryl, where each of the alkyl, cycloalkyl, heterocyclyl, and aryl is independently optionally substituted.

22. A method according to any one of claims 1 to 21 wherein the patient is previously identified as having or at risk of a cancer characterized by 5-LO expression.

23. A method according to any one of claims 1 to 21 further comprising identifying the patient as having or at risk of a cancer characterized by 5-LO expression before said administration.

24. A method according to any one of claims 1 to 23 wherein one or more cancer cells in the patient have increased expression of 5-LO relative to control cells.

25. A method according to any one of claims 1 to 24 wherein the expression of 5-LO in one or more cancer cells in the patient is greater than a predetermined threshold value.

26. A method according to any one of claims 1 to 25 wherein the cancer is further characterised by NAD(P)H:quinone oxireductase 1 (NQO1) expression.

27. A method according to claim 26 wherein the patient is previously identified as having or at risk of a cancer characterized by 5-LO and NQO1 expression.

28. A method according to claim 26 further comprising identifying the patient as having or at risk of a cancer characterized by 5-LO and NQO1 expression before said administration.

29. A method according to any one of claims 26 to 28 wherein one or more cancer cells in the patient have increased expression of 5-LO and NQO1 relative to control cells.

30. A method according to any one of claims 26 to 29 wherein the expression of 5-LO and NQO1 in one or more cancer cells in the patient is greater than a predetermined threshold value.

31. A method according to any one of claims 1 to 30 wherein cancer is a blood cancer.

32. A method according to claim 31 wherein the blood cancer is AML or CLL.

33. A method according to any one of claims 1 to 32 wherein the β-lapachone compound is administered in combination with a second therapeutic agent.

34. A method according to claim 33 wherein the second therapeutic agent is an anti-cancer compound.

35. A method according to claim 34 wherein the anti-cancer compound is selected from an anthracycline, gemcitarabine, cytarabine, vincristine, L-asparaginase, cyclophosphamide, fibromun, dacarbazine, methotrexate and 6-mercaptopurine, chlorambucil, an alkylating agent, cyclophosphamide, corticosteroids, imatinib, cladribine, pentostatin, rituximab, chlorambucil, a taxane, and doxorubicin.

36. A method according to any one of claims 1 to 32 wherein the β-lapachone compound is administered in combination with irradiation.

37. A β-lapachone compound for use in a method of treating cancer characterized by 5-lipoxygenase (5-LO) expression according to any one of claims 1 to 36.

38. Use of the β-lapachone compound in the manufacture of a medicament for use in a method of treating cancer characterized by 5-lipoxygenase (5-LO) expression according to any one of claims 1 to 36.

39. A method of selecting a cancer patient for treatment with a β-lapachone compound comprising
providing a sample of cancer cells from a cancer patient,
determining the presence of 5-LO expression in the cancer cells, and
selecting a cancer patient with cancer cells that express 5-LO for treatment with the β-lapachone compound.

40. A method of prognosis of a cancer patient comprising
providing a sample of cancer cells from a cancer patient, and,
determining the level of 5-LO expression in the cancer cells, the level of 5-LO expression being indicative of the prognosis of the patient.

41. A method according to claim 39 or claim 40 wherein the cancer is a blood cancer

42. A method according to claim 41 wherein the blood cancer is CLL or AML.
